SEQUENCE LISTING (14 sequences)

SEQ ID NO: 1 - DNA, 1278 bp, Artificial
  Codon-optimized (for expression in E. coli) nucleotide sequence encoding P13G11
atggcaaaaaagctgcctaaagtgaaaggtctggacaacaccgtggacatcatcaagggtggctatacctacgtgcctggcaaactggaggagttcgacagcaaagcattcgaagtgcgcgccctgggtggcaagaagatcgcagtgatgagcggcaaggaagccgccgagattttttatgataacgaaaaaatggagcgtcagggtaccctgccgaagcgcatcgtgaacacactgttcggtaaaggcgccattcataccaccgccggcaagaaacatgtggatcgcaaggcactgttcatgagcctgatgaccgagggcaatctgaattacgtgcgcgaactgacgcgtacgctgtggcatgccaatacgcagcgcatggaaagcatggacgaggtgaacatctaccgcgagtcgattgtgctgctgaccaaagttggcacccgctgggcgggcgttcaggcgccgccggaggatattgaacgcattgcgaccgacatggacatcatgatcgatagcttccgcgcgttaggcggcgcgttcaaaggctacaaagcgtcgaaggaggcgcgtcgccgtgtggaagattggctggaggagcagatcatcgagacccgcaaaggcaacattcacccgccggaaggtaccgcgctgtacgaattcgcccattgggaagacctgaacgataacccgatggacagccatctgtgcgccgttgatctgatgaacgttgttcgcccgctggcagcaattaaccgcttcattagctacggcgttaaagtgctgatcgaattcgaccaggaaaaagaaaagctgcgcctggagaacaacgaggactacgcctacaagttcgcacaggaagtgcgccgtatctttccgttcgtgccttacttaccgggtcgcgccgccgtggatctggagtatgatggctataagatcccggccggtatgatgaccgccctggatgtttacggtaccacacacgatgaggatctgtgggagaatccggatcagttcaacccgaatcgttttgataactgggacggcagtccgtttgatctgattccgcagggcggtggcgatttctacaccaatcatcgttgcgccggcgagtggatcaccgtgattattatggaagaaacaatgaaatactttgccaacaaaattgaattcgatgtgccgagtcaggacctgagcgttaaactggacaaactgcctggcaacgtgaccagcggtaccatcattagcaacgtgcgtccgcgtgttgcccgcaaataa

SEQ ID NO: 2 - protein, 425 aa, Artificial
  P13G11
MAKKLPKVKGLDNTVDIIKGGYTYVPGKLEEFDSKAFEVRALGGKKIAVMSGKEAAEIFYDNEKMERQGTLPKRIVNTLFGKGAIHTTAGKKHVDRKALFMSLMTEGNLNYVRELTRTLWHANTQRMESMDEVNIYRESIVLLTKVGTRWAGVQAPPEDIERIATDMDIMIDSFRALGGAFKGYKASKEARRRVEDWLEEQIIETRKGNIHPPEGTALYEFAHWEDLNDNPMDSHLCAVDLMNVVRPLAAINRFISYGVKVLIEFDQEKEKLRLENNEDYAYKFAQEVRRIFPFVPYLPGRAAVDLEYDGYKIPAGMMTALDVYGTTHDEDLWENPDQFNPNRFDNWDGSPFDLIPQGGGDFYTNHRCAGEWITVIIMEETMKYFANKIEFDVPSQDLSVKLDKLPGNVTSGTIISNVRPRVARK

SEQ ID NO: 3 - DNA, 1278 bp, Artificial
  Codon-optimized (for expression in E. coli) nucleotide sequence encoding P3D3
atggcaaaaaagctgcctaaagtgaaaggtctggacaacaccgtggacatcatcaagggtggctatacctacgtgcctggcaaactggaggagttcgacagcaaagcattcgaagtgcgcgccctgggtggcaagaagatcgcagtgatgagcggcaaggaagccgccgagattttttatgataacgaaaaaatggagcgtcagggtaccctgccgaagcgcatcgtgaacacactgttcggtaaaggcgccattcataccaccgccggcaagaaacatgtggatcgcaaggcactgttcatgagcctgatgaccgagggcaatctgaattacgtgcgcgaactgacgcgtacgctgtggcatgccaatacgcagcgcatggaaagcatggacgaggtgaacatctaccgcgagtcgattgtgctgctgaccaaagttggcacccgctgggcgggcgttcaggcgccgccggaggatattgaacgcattgcgaccgacatggacatcatgatcgatagcttccgcgcgttaggcggcgcgttcaaaggctacaaagcgtcgaaggaggcgcgtcgccgtgtggaagattggctggaggagcagatcatcgagacccgcaaaggcaacattcacccgccggaaggtaccgcgctgtacgaattcgcccattgggaagactatctgggcaacccgatggacagccatctgtgcgccgttgatctgatgaacgttgttcgcccgctggcagcaattaaccgcttcattagctacggcgttaaagtgctgatcgaattcgaccaggaaaaagaaaagctgcgcctggagaacaacgaggactacgcctacaagttcgcacaggaagtgcgccgtatctttccgttcgtgccttacttaccgggtcgcgccgccgtggatctggagtatgatggctataagatcccggccggtatgatgaccgccctggatgtttacggtaccacacacgatgaggatctgtgggagaatccggatcagttcaacccgaatcgttttgataactgggacggcagtccgtttgatctgattccgcagggcggtggcgatttctacaccaatcatcgttgcgccggcgagtggatcaccgtgattattatggaagaaacaatgaaatactttgccaacaaaattgaattcgatgtgccgagtcaggacctgagcgttaaactggacaaactgcctggcaacgtgaccagcggtaccatcattagcaacgtgcgtccgcgtgttgcccgcaaataa

SEQ ID NO: 4 - protein, 425 aa, Artificial
  P3D3
MAKKLPKVKGLDNTVDIIKGGYTYVPGKLEEFDSKAFEVRALGGKKIAVMSGKEAAEIFYDNEKMERQGTLPKRIVNTLFGKGAIHTTAGKKHVDRKALFMSLMTEGNLNYVRELTRTLWHANTQRMESMDEVNIYRESIVLLTKVGTRWAGVQAPPEDIERIATDMDIMIDSFRALGGAFKGYKASKEARRRVEDWLEEQIIETRKGNIHPPEGTALYEFAHWEDYLGNPMDSHLCAVDLMNVVRPLAAINRFISYGVKVLIEFDQEKEKLRLENNEDYAYKFAQEVRRIFPFVPYLPGRAAVDLEYDGYKIPAGMMTALDVYGTTHDEDLWENPDQFNPNRFDNWDGSPFDLIPQGGGDFYTNHRCAGEWITVIIMEETMKYFANKIEFDVPSQDLSVKLDKLPGNVTSGTIISNVRPRVARK

SEQ ID NO: 5 - DNA, 1275 bp, Artificial
  codon-optimized nucleotide sequence encoding Sm46-del29
gcaaaaaagctgcctaaagtgaaaggtctggacaacaccgtggacatcatcaagggtggctatacctacgtgcctggcaaactggaggagttcgacagcaaagcattcgaagtgcgcgccctgggtggcaagaagatcgcagtgatgagcggcaaggaagccgccgagattttttatgataacgaaaaaatggagcgtcagggtaccctgccgaagcgcatcgtgaacacactgttcggtaaaggcgccattcataccaccgccggcaagaaacatgtggatcgcaaggcactgttcatgagtctgatgaccgatgaaaatttaaattatctgcgcgaactgacacgcaactattggtttatgaatacagaacgcatgcagagcatggataaagtgaatgtgtacaatgaaagcatttatatgctgaccaaaattggcttccgctgggccggtatcattcagacccctgaagaggccgagcagaatgccaaagacatggacaccatgatcaacagctttgtgagcctgggcagcgcctacaagggttacaaaaaagccaagaaagcccgcaagcgcgtggaagattttctggagaaacaaattatcgacgttcgtaaaggcaaactgcatccggaggaaggtaccgccctgtacgaattcgcccattgggaagacctgaacgataacccgatggacagccatctgtgcgccgttgatctgatgaacgttgttcgcccgctggcagcaattaaccgcttcattagctacggcgttaaagtgctgatcgaattcgaccaggaaaaagaaaagctgcgcctggagaacaacgaggactacgcctacaagttcgcacaggaagtgcgccgtatctttccgttcgtgccttacttaccgggtcgcgccgccgtggatctggagtatgatggctataagatcccggccggtatgatgaccgccctggatgtttacggtaccacacacgatgaggatctgtgggagaatccggatcagttcaacccgaatcgttttgataactgggacggcagtccgtttgatctgattccgcagggcggtggcgatttctacaccaatcatcgttgcgccggcgagtggatcaccgtgattattatggaagaaacaatgaaatactttgccaacaaaattgaattcgatgtgccgagtcaggacctgagcgttaaactggacaaactgcctggcaacgtgaccagcggtaccatcattagcaacgtgcgtccgcgtgttgcccgcaaataa

SEQ ID NO: 6 - protein, 424 aa, Artificial
  Sm46-del29
AKKLPKVKGLDNTVDIIKGGYTYVPGKLEEFDSKAFEVRALGGKKIAVMSGKEAAEIFYDNEKMERQGTLPKRIVNTLFGKGAIHTTAGKKHVDRKALFMSLMTDENLNYLRELTRNYWFMNTERMQSMDKVNVYNESIYMLTKIGFRWAGIIQTPEEAEQNAKDMDTMINSFVSLGSAYKGYKKAKKARKRVEDFLEKQIIDVRKGKLHPEEGTALYEFAHWEDLNDNPMDSHLCAVDLMNVVRPLAAINRFISYGVKVLIEFDQEKEKLRLENNEDYAYKFAQEVRRIFPFVPYLPGRAAVDLEYDGYKIPAGMMTALDVYGTTHDEDLWENPDQFNPNRFDNWDGSPFDLIPQGGGDFYTNHRCAGEWITVIIMEETMKYFANKIEFDVPSQDLSVKLDKLPGNVTSGTIISNVRPRVARK

SEQ ID NO: 7 - protein, 25 aa, Artificial
  Enzyme fragment 1
TLWHANTQRMESMDEVNIYRESIVL

SEQ ID NO: 8 - protein, 96 aa, Artificial
  Enzyme fragment 2
TLWHANTQRMESMDEVNIYRESIVLLTKVGTRWAGVQAPPEDIERIATDMDIMIDSFRALGGAFKGYKASKEARRRVEDWLEEQIIETRKGNIHPP

SEQ ID NO: 9 - protein, 112 aa, Artificial
  Enzyme fragment 3
TLWHANTQRMESMDEVNIYRESIVLLTKVGTRWAGVQAPPEDIERIATDMDIMIDSFRALGGAFKGYKASKEARRRVEDWLEEQIIETRKGNIHPPEGTALYEFAHWEDYLG

SEQ ID NO: 10 - protein, 425 aa, Artificial
  Amino acid sequence of P26H9
MAKKLPKVKGLDNTVDIIKGGYTYVPGKLEEFDSKAFEVRALGGKKIAVMSGKEAAEIFYDNEKMERQGTLPKRIVNTLFGKGAIHTTAGKKHVDRKALFMSLMTDENLNYLRELTRNYWFMNTERMQSMDKVNVHNESIYMLTKIGFRWAGIIQTPEEAEQNAKDMDTMINSFVSLGSAYKGYKKAKKARKRVEDFLEKQIIDVRKGKLHPEEGTALYEFAHWEDLNDNPMDSHLCAVDLMNVVRPLAAINRFISYGVKVLIEFDQEKEKLRLENNEDYAYKFAQEVRRIFPFVPYLPGRAAVDLEYDGYKIPAGMMTALDVYGTTHDEDLWENPDQFNPNRFDNWDGSPFDLIPQGGGDFYTNHRCAGEWITVIIMEETMKYFANKIEFDVPSQDLSVKLDKLPGNVTSGTIISNVRPRVARK

SEQ ID NO: 11 - protein, 425 aa, Artificial
  Amino acid sequence of P40E6
MAKKLPKVKGLDNTVDIIKGGYTYVPGKLKEFDSKAFEVRALGGKKIAVMSGKEAAEIFYDNEKMERQGTLPKRIVNTLFGKGAIHTTAGKKHVDRKALFMSLMTDENLNYLRELTRNYWFMNTERMQSMDKVNVYNESIYMLTKIGFRWAGIIQTPEEAEQNAKDMDTMINSFVSLGSAYKGYKKAKKARKRVEDFLEKQIIDVRKGKLHPEEGTALYEFAHWEDLNDNPMDSHLCAVDLMNVVRPLAAINRFISYGVKVLIEFDQEKEKLRLENNEDYAYKFAQEVRRIFPFVPYLPGRAAVDLEYDGYKIPAGMMTALDVYGTTHDEDLWENPDQFNPNRFDNWDGSPFDLIPQGGGDFYTNHRCAGEWITVIIMEETMKYFANKIEFDVPSQDLSVKLDKLPGNVTSGTIISNVRPRVARK

SEQ ID NO: 12 - protein, 425 aa, Artificial
  Amino acid sequence of P42E11
MAKKLPKVKGLDNTVDIIKGGYTYVPGKLEEFDSKAFEVRALGGKKIAVMSGKEAAEIFYDNEKMERQGTLPKRIVNTLFGKGAIHTTAGKKHVDRKALFMSLMTDENLNYLRELTRNYWFMNTERMQSMDKVNVYNESIYMLTKIGFRWAGIIQTPEEAEQNAKDMDTMINSFVSLGSAYKGYKKAKKARKRVEDFLEKQIIDVRKGKLHPEEGTALYEFAHWEDLNDNPMDSHLCAVDLINVVRPLAAINRFISYGVKVLIEFDQEKEKLRLENNEDYAYKFAQEVRRIFPFVPYLPGRAAVDLEYDGYKIPAGMMTALDVYGTTHDEDLWENPDQFNPNRFDNWDGSPFDLIPQGGGDFYTNHRCAGEWITVIIMEETMKYFANKIEFDVPSQDLSVKPDKLPGNVTSGTIISNVRPRVARK

SEQ ID NO: 13 - protein, 425 aa, Artificial
  Amino acid sequence of P21G12
MAKKLPKVKGLDNTVDIIKGGYTYVPGKLEEFDSKAFEVRALGGKKIAVMSGKEAAEIFYDNEKMERQGTLPKRIVNTLFGKGAIHTTAGKKHVDRKALFMSLMTDENLNYLRELTRNYWFMNTERMQSMDKVNVYNESIYMLTKIGFRWAGIIQTPEEAEQNAKDMDTMINSFVSLGSAYKGYKKAKKARKRVEDFLEKQIIDVRKGKLHPEEGTALYEFAHWEDYLGNPMDSHLCAVDLMNVVRPLAAINRFISYGVKVLIEFDQEKEKLRLENNEDYAYKFAQEVRRIFPFVPYLPGRAAVDLEYDGYKIPAGMMTALDVYGTTHDEDLWENPDQFNPNRFDNWDGSPFDLIPQGGGDFYTNHRCAGEWITVIIMEETMKYFANKIEFDVPSQDLSVKLDKLPGNVTSGTIISNVRPRVARK

SEQ ID NO: 14 - protein, 425 aa, Artificial
  Amino acid sequence of P41A3
MAKKLPKVKGLDNTVDIIKGGYTYVPGKLEEFDSKAFEVRALGGKKIAVMSGKEAAEIFYDNEKMERQGTLPKRIVNTLFGKGAIHTTAGKKHVDRKALFMSLMTDENLNYLRELTRNYWFMNTERMQSMDKVNVYNESIYMLTKIGFRWAGIIQTPEEAEQNAKDMDTMINSFVSLGSAYKGYKKAKKARKRVEDFLEKQIIDVRKGKLHPEEGTALYEFAHWEDLNDNPMDSHLCAVDLMNVVRPLAAINRFISYGVKVLIEFDQEKEKLRLENNEDYAYKFAQEVRRIFPFVPYLPGRAAVDLEYDGYKIPAGMMTALDVYGTTHDESLWDDPNEFRPERFETWDGSPFDLIPQGGGDFYTNHRCAGEWITVIIMEETMKYFAEKIKYDVPEQDLEVDLNSIPGYVKSGFVIKNVREVVDRT

(19) United States
(12) Patent Application Publication
(10) Pub. No.: US 2021/0292733 A1
(43) Pub. Date: Sep. 23, 2021

(54) IMPROVED CYTOCHROME P450 FATTY ACID DECARBOXYLASES

(71) Applicants: TOTAL RAFFINAGE CHIMIE, Courbevoie (FR); QINGDAO INSTITUTE OF BIOENERGY AND BIOPROCESS TECHNOLOGY (QIBEBT), Qingdao, Shandong (CN)

(72) Inventors: Shengying LI, Qingdao, Shandong (CN); Huifang XU, Qingdao, Shandong (CN); Linlin NING, Qingdao, Shandong (CN); Laurent FOURAGE, Suresnes (FR)

(73) Assignees: TOTAL RAFFINAGE CHIMIE, Courbevoie (FR); QINGDAO INSTITUTE OF BIOENERGY AND BIOPROCESS TECHNOLOGY (QIBEBT), Qingdao, Shandong (CN)

(21) Appl. No.: 17/260,354

(22) PCT Filed: Jul. 25, 2019
(86) PCT No.: PCT/EP2019/070000

§ 371 Date: Jan. 14, 2021

(30) Foreign Application Priority Data
Jul. 25, 2018 (EP) 18290089.4

(51) Int. Cl. C12N 9/88; C12P 5/02; C10M 105/04

(52) U.S. Cl. CPC C12N 9/88; C10M 105/04; C12P 5/026

(57) ABSTRACT

The present invention relates to biocatalysts catalyzing the formation of α-olefins. In particular, the invention provides polypeptides with improved decarboxylase activity on C₁₀-C₁₆ free fatty acids, more particularly on C₁₀ or C₁₂ free fatty acids, as compared to the P450 fatty acid decarboxylase isolated from the Staphylococcus massiliensis strain S46 (Sm46). The invention further provides recombinant nucleic acids and vectors comprising the coding sequences encoding these polypeptides, genetically engineered host cells expressing said polypeptides and methods for the production of C₉-C₁₅ α-olefins, more particularly C₉ or C₁₁ α-olefins, using said polypeptides or said host cells.

### TECHNICAL FIELD

The application generally relates to biocatalysts. In particular, the application relates to improved P450 fatty acid decarboxylases catalyzing the formation of α-olefins.

### BACKGROUND

Enzymes isolated from micro-organisms represent a natural resource of biocatalysts useful in industry. However, their catalytic activities are often relatively moderate and/or their substrate preference/selectivity sub-optimal for the industrial process of interest.

The P450 fatty acid decarboxylase isolated and identified from the Staphylococcus massiliensis strain S46 exhibits moderate fatty acid (FA) decarboxylation activity towards mid-chain length free fatty acids (WO2017001606), thereby forming terminal olefin products that could be valuable biofuel molecules or precursors of lubricants or surfactants. However, this P450 fatty acid decarboxylase also catalyzes hydroxylation of fatty acids as side reactions. Consequently, fatty acid decarboxylation reactions are accompanied by tangible fatty acid hydroxylation side-reactions, thereby generating unwanted hydroxyl-fatty acid products.

In order to efficiently produce α-alkenes, e.g. as alternative biofuels, there is a need for enzymes with predominant decarboxylation activity, in particular decarboxylation activity towards mid-chain free fatty acids (FFAs).

Previously, attempts have been made to apply site-directed mutagenesis and rational or semi-rational design approaches on the P450 fatty acid decarboxylase OleTJE and another P450 peroxygenase from Methylobacterium populi that showed marginal fatty acid decarboxylation activity (Wang et al. 2016 Chem Commun (Camb) 52: 8131-8133; Amaya et al. 2016 J lnorg Biochem 158:11-16; Xu et al. 2017 Biotechnology for Biofuels 10:208; Fang et al. 2017 Scientific Reports 7:44258; Matthews et al. 2017 J Biol Chem 292:5128-5143; Amaya et al. 2018 Biochemistry 57:344-353). These efforts did either not result in an improved fatty acid conversion rate or resulted in only a negligible improvement, or even diminished fatty acid decarboxylation activity in favor of fatty acid hydroxylation activity.

Accordingly, there remains a need for enzymes with predominant decarboxylation activity, in particular decarboxylation activity towards C₁₀ to C₁₆ free fatty acids. It is also desired that the enzymes have an increased substrate conversion and α-olefin production rate, preferably when used at low concentration.

### SUMMARY OF THE INVENTION

The present invention solves one or more of the above described problems of the prior art. In particular, novel cytochrome P450 fatty acid decarboxylases are provided that show high decarboxylation activity towards C₁₀ to C₁₆ free fatty acids, more particularly on C₁₀ or C₁₂ free fatty acids. More particularly, the enzymes show a high conversion rate of C₁₀ to C₁₆ free fatty acids to the corresponding α-olefins, and thus a high C₉ to C₁₅ α-olefin production. In particular, the novel cytochrome P450 fatty acid decarboxylases show improved decarboxylation activity towards C₁₀ to C₁₆ free fatty acids compared to known decarboxylases such as the P450 fatty acid decarboxylase isolated from the Staphylococcus massiliensis strain S46 (Sm46) or the cytochrome P450 from Methylobacterium populi ATCC BAA 705 (CYP-MP). The enzymes show an improved conversion rate of C₁₀ to C₁₆ free fatty acids and improved C₉ to C₁₅ α-olefin production compared to known decarboxylases such as P450 fatty acid decarboxylase Sm46 or CYP-MP. For example, the decarboxylases ensure an improved conversion rate of C₁₂ free fatty acids and an increased C₁₁ α-olefin production. The novel decarboxylases also show a higher ratio of decarboxylation activity over hydroxylation activity. These improved cytochrome P450 fatty acid decarboxylases are also shown to possess faster catalytic rates (i.e. faster conversion of the free fatty acid substrates and faster α-olefin production) at lower enzyme concentrations.

The present invention is in particular captured by any one or any combination of one or more of the below numbered aspects and embodiments (i) to (xvii):

(i) A polypeptide having fatty acid decarboxylase activity, wherein said polypeptide comprises an amino acid sequence having at least 95%, preferably at least 99%, identity to SEQ ID NO:4 or SEQ ID NO:2.
(ii) The polypeptide according to (i), wherein said polypeptide converts at least 2.0 times more C₁₂ free fatty acids to C₁₁ α-olefins as compared to the enzyme set forth in SEQ ID NO:6.
(iii) The polypeptide according to (i) or (ii), wherein said polypeptide converts at least 10% more C₁₂ (free) fatty acid substrate in vitro as compared to the enzyme set forth in SEQ ID NO:6.
(iv) A recombinant nucleic acid comprising a nucleotide sequence encoding the polypeptide according to any one of (i) to (iii).
(v) The recombinant nucleic acid according to (iv) comprising a nucleotide sequence as set forth in SEQ ID NO:3 or SEQ ID NO:1.
(vi) A vector comprising the recombinant nucleic acid according to (iv) or (v).
(vii) A host cell comprising the recombinant nucleic acid according to (iv) or (v) or the vector according to (vi).
(viii) The host cell according to (vii), which is an oleaginous host cell, preferably an oleaginous alga or an oleaginous yeast.
(ix) A method for the production of α-olefins comprising culturing a host cell according to (vii) or (viii) under conditions suitable for the production of α-olefins by said host cell.
(x) The method according to (ix), wherein said α-olefins are C₉-C₁₅ α-olefins, preferably C₉ or C₁₁ α-olefins.
(xi) The method according to any one of (ix) or (x), wherein said host cell is cultivated in medium comprising C₁₀-C₁₆ free fatty acids, preferably C₁₀ or C₁₂ free fatty acids.
(xii) The method according to any one of (ix) to (xi), wherein the host cell has further been genetically engineered to produce or overproduce C₁₀-C₁₆ free fatty acids, preferably C₁₀ or C₁₂ free fatty acids, preferably wherein the host cell comprises a recombinant nucleic acid encoding an enzyme involved in the production of free fatty acids with a carbon chain length comprised between 10 and 16 such as a thioesterase having activity on C₁₀ to C₁₆ acyl-ACP.
(xii) The method according to any one of (ix) to (xii), further comprising the step of recovering the α-olefins from the host cell or the culture medium.
(xiv) A method for the production of α-olefins comprising contacting a polypeptide according to any one of (i) to (iii) with free fatty acids, preferably C₁₀-C₁₆ free fatty acids, more preferably C₁₀ or C₁₂ free fatty acids.
(xv) A method for the production of poly-α-olefins comprising the following steps:
i) producing α-olefins according to a method according to any one of (ix) to (xiii); and
ii) performing an oligomerization reaction using the α-olefins obtained in step i) as monomer to produce an oligomer; and optionally,
iii) hydrogenating the oligomer produced in step ii).
   (xvi) The method according to (xv), wherein the poly-α-olefins are C₂₇ or C₃₃ poly-α-olefins, wherein step i) comprises the production of C₉ or C₁₁ α-olefins, and wherein the oligomerization reaction in step ii) is a trimerization reaction.
   (xvii) Use of a polypeptide according to any one of (i) to (iii) or a host cell according to (vii) or (viii) for the industrial production of lubricants.

### BRIEF DESCRIPTION OF THE FIGURES

The teaching of the application is illustrated by the following Figures which are to be considered as illustrative only and do not in any way limit the scope of the claims.

FIG. 1: Nucleotide (A,C,E) and amino acid (B,D,F-K) sequences of novel decarboxylase enzymes P13G11 (A,B), P3D3 (C,D), P26H9 (G), P40E6 (H), P42E11 (I), P21G12 (J) and P41A3 (K) according to particular embodiments of the invention, and the decarboxylase enzyme Sm46Δ29 (E,F).

FIG. 2: GC analysis of the lauric acid conversion (C12) and α-undecene production (C11) by Sm46Δ29 and novel decarboxylase enzymes according to particular embodiments of the invention. C11 (left bars) represents α-undecene production and C12 (right bars) represents the remaining lauric acid substrate, both indicated as the area under the gas chromatographic (GC) peak. The reaction mixtures contained 200 μM C₁₂ fatty acid substrate, 220 μM H₂O₂, and 0.5 μM of the indicated enzyme or no enzyme (C12 Std.) in a total volume of 200 μl, and were incubated for 40 min at 30° C. Experiments were done in duplicates.

FIG. 3: Substrate specificity of the novel decarboxylase enzyme P13G11 according to a particular embodiment of the invention. 200 μl reaction mixtures containing 200 μM of each fatty acid substrate, 220 μM H₂O₂ and 2 μM of the purified P13G11 enzyme were incubated at 30° C. for 2 hours. Conversion percentages of the free fatty acid substrates (right bars) and the corresponding α-alkene products (left bars) are shown. Experiments were done in duplicates. One of the two independent sets of experiments is shown.

FIG. 4: Comparison of the decarboxylation and hydroxylation activities against C₁₂ fatty acid substrate by Sm46Δ29 (Sm46) enzyme and the novel decarboxylase enzyme P13G11 at different enzyme concentrations (0.5 μM v.s. 2.0 μM as indicated) according to particular embodiments of the invention. All reaction mixtures contained 200 μM of C₁₂ fatty acid substrate, 220 μM H₂O₂ and the indicated amount of purified enzyme, and were incubated at 30° C. for 40 min (A) or for 2 h (B). The ratios of decarboxylation activity (lower part of the bars) over hydroxylation activity (upper part of the bars) are shown above the columns.

FIG. 5: Reaction rates of the Sm46Δ29 (Sm46) enzyme and the novel decarboxylase enzyme P13G11 for C₁₂ lauric acid conversion and C_{11 a}-olefin production. All reactions were carried out at 30° C. in 200 μl Na—PO₄ buffer (pH 7.4) containing 200 μM C₁₂ fatty acid substrate, 0.5 μM (A, B) or 2 μM (C, D) of the indicated enzyme and 220 μM H₂O₂ for the indicated times. Data shown are the percentage of C₁₂ fatty acid substrate conversion (A,C) and the percentage of C₁₁ olefin production (B,D).

FIG. 6: Reaction rates of novel decarboxylase enzymes P13G11 and P3D3 for C₁₂ lauric acid conversion and C_{11 a}-olefin production according to particular embodiments of the invention. All reactions were carried out at 30° C. in 200 μl Na—PO₄ buffer (pH 7.4) containing 200 μM C₁₂ fatty acid substrate, 0.5 μM of the indicated enzyme and 220 μM H₂O₂ for the indicated times. Data shown are the percentage of C₁₂ fatty acid substrate conversion (A) and the percentage of C₁₁ olefin production (B).

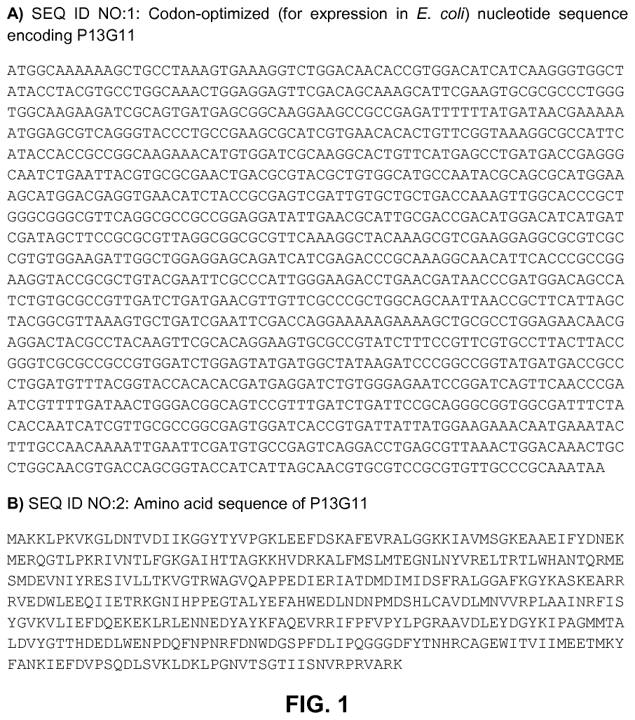

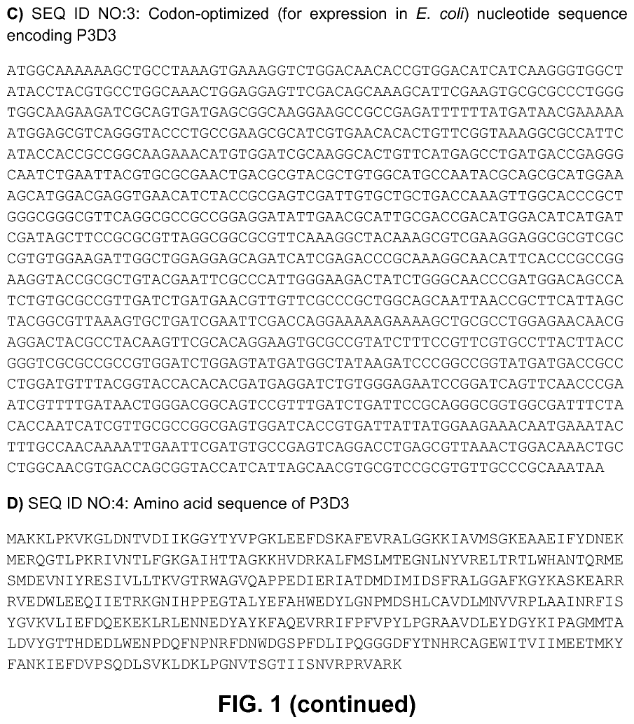

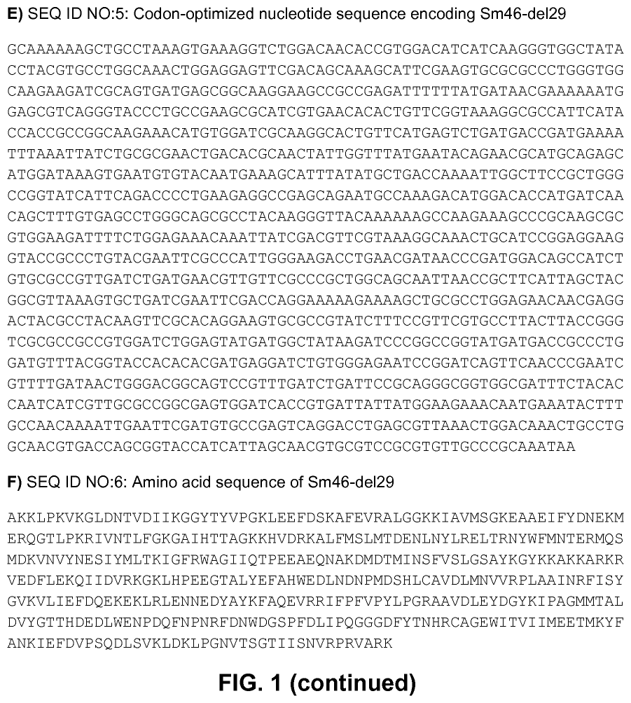

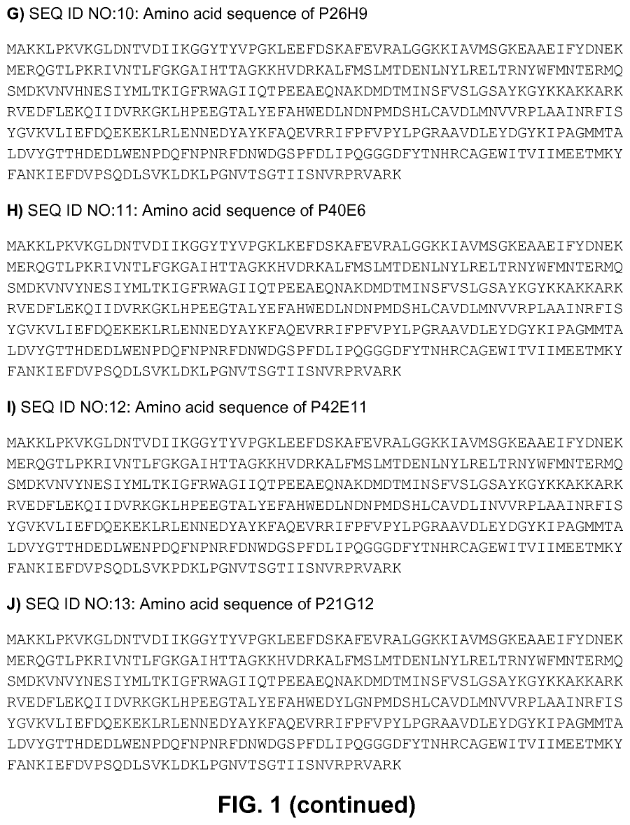

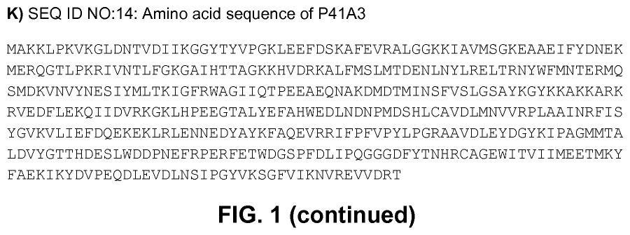

### DETAILED DESCRIPTION OF THE INVENTION

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. Where reference is made to embodiments as comprising certain elements or steps, this encompasses also embodiments which consist essentially of the recited elements or steps.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

All documents cited in the present specification are hereby incorporated by reference in their entirety.

Standard reference work setting forth the general principles of biochemistry includes Biochemical Pathways: An Atlas of Biochemistry and Molecular Biology, ed. Michal, G, John Wiley and Sons, Inc., New York, US, 1999.

The term “olefin” or “alkene” refers herein to molecules composed of carbon and hydrogen, containing at least one carbon-carbon double bond. Olefins containing one carbon-carbon double bond are denoted herein as mono-unsaturated hydrocarbons and have the chemical formula CₙH₂ₙ, where n equals at least two.

“Alpha-olefins”, “α-olefins”, “1-alkenes” or “terminal olefins” are used as synonyms herein and denote olefins or alkenes having a double bond at the primary or alpha (α) position. “Linear α-olefins” or “LAO” as used herein refer to α-olefins that have a linear hydrocarbon chain, whereas “branched α-olefins” have a branch on one or more carbon atoms of the hydrocarbon chain. The term “C₉ to C₁₅ α-olefins” is used herein to denote α-olefins with 9 to 15 carbons and encompasses any one or more of C₉ α-olefins, C₁₀ α-olefins, C₁₁ α-olefins, C₁₂ α-olefins, C₁₃ α-olefins, C₁₄ α-olefins, and C₁₅ α-olefins. The term “uneven-numbered α-olefins” refers to α-olefins wherein the number of carbon atoms is not even in number. Thus, uneven-numbered C₉ to C₁₅ α-olefins encompass C₉, C₁₁, C₁₃ and C₁₅ α-olefins.

As used herein, the term “fatty acid” or “free fatty acid” means a carboxylic acid having the formula RCOOH, or a salt (RCOO—) thereof. R represents an aliphatic group, preferably an alkyl group. Fatty acids can be saturated, mono-unsaturated, or poly-unsaturated. The term “C₁₀ to C₁₆ fatty acids” or “C₁₀ to C₁₆ free fatty acid” as used herein denotes a fatty acid or free fatty acid having 10 to 16 carbon atoms and encompasses any one or more of C₁₀ fatty acid or free fatty acids, C₁₁ fatty acid or free fatty acids, C₁₂ fatty acid or free fatty acids, C₁₃ fatty acid or free fatty acids, C₁₄ fatty acid or free fatty acids, C₁₅ fatty acid or free fatty acids, and C₁₆ fatty acid or free fatty acids. The term “even-numbered fatty acids” refers to fatty acids wherein the number of carbon atoms is even in number. Thus, even-numbered C₁₀ to C₁₆ fatty acids encompass C₁₀, C₁₂, C₁₄ and C₁₆ fatty acids.

As used herein, the term “host cell” refers to a cell that can be used to produce an α-olefin as described herein. A host cell may be an isolated cell or a cell line grown in culture, or a cell which resides in a living tissue or organism.

As used herein, the terms “microbial”, “microbial organism” or “micro-organism” are intended to mean any organism that exists as a microscopic cell that is included within the domains of archaea, bacteria or eukaryotes. Therefore, the term is intended to encompass prokaryotic or eukaryotic cells or organisms having a microscopic size and includes bacteria, archaea and eubacteria such as cyanobacteria of all species as well as eukaryotic micro-organisms such as fungi, including yeasts, and algae. The term also includes cell cultures of any species that can be cultured for the production of a biochemical.

The term “oleaginous” as used herein with reference to a host cell denotes cells characterized by their lipid accumulation capability. Typically, their biomass contains over 20% lipids in dry matter.

The “algae” group encompasses, without limitation, (i) several eukaryotic phyla, including the Rhodophyta (red algae), Chlorophyta (green algae), Dinoflagellata, Haptophyta, (ii) several classes from the eukaryotic phylum Heterokontophyta which include without limitation the classes Bacillariophycea (diatoms), Eustigmatophycea, Phaeophyceae (brown algae), Xanthophyceae (yellow-green algae) and Chrysophyceae (golden algae), and (iii) the prokaryotic phylum Cyanobacteria (blue-green algae).

The term “algae” includes for example genera selected from: Achnanthes, Amphora, Anabaena, Anikstrodesmis, Arachnoidiscusm, Aster, Botryococcus, Chaetoceros, Chlamydomonas, Chlorella, Chlorococcum, Chorethron, Cocconeis, Coscinodiscus, Cyclotella, Cylindrotheca, Dunaliella, Emiliana, Euglena, Fistulifera, Fragilariopsis, Gyrosigma, Hematococcus, Isochrysis, Lampriscus, Monochrysis, Monoraphidium, Nannochloris, Nannnochloropsis, Navicula, Neochloris, Nephrochloris, Nephroselmis, Nitzschia, Nodularia, Nostoc, Odontella, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

The terms “genetically engineered” or “genetically modified” or “recombinant” as used herein with reference to a host cell denote a non-naturally occurring host cell, as well as its recombinant progeny, that has at least one genetic alteration not found in a naturally occurring strain of the referenced species, including wild-type strains of the referenced species. Such genetic modification is typically achieved by technical means (i.e. non-naturally) through human intervention and may include, e.g., the introduction of an exogenous nucleic acid and/or the modification, over-expression, or deletion of an endogenous nucleic acid.

The term “exogenous” or “foreign” as used herein is intended to mean that the referenced molecule, in particular nucleic acid, is not naturally present in the host cell. The term “endogenous” or “native” as used herein denotes that the referenced molecule, in particular nucleic acid, is present in the host cell.

By “recombinant nucleic acid” when referring to a nucleic acid in a recombinant host cell, is meant that at least part of said nucleic acid is not naturally present in the host cell in the same genomic location. For instance a recombinant nucleic acid can comprise a coding sequence naturally occurring in the host cell under control of an exogenous promotor, or it can be an additional copy of a gene naturally occurring in the host cell, or a recombinant nucleic acid can comprise an exogenous coding sequence under the control of an endogenous promoter.

By “nucleic acid” is meant oligomers and polymers of any length composed essentially of nucleotides, e.g., deoxyribonucleotides and/or ribonucleotides. Nucleic acids can comprise purine and/or pyrimidine bases and/or other natural (e.g., xanthine, inosine, hypoxanthine), chemically or biochemically modified (e.g., methylated), non-natural, or derivatised nucleotide bases. The backbone of nucleic acids can comprise sugars and phosphate groups, as can typically be found in RNA or DNA, and/or one or more modified or substituted sugars and/or one or more modified or substituted phosphate groups. Modifications of phosphate groups or sugars may be introduced to improve stability, resistance to enzymatic degradation, or some other useful property. A “nucleic acid” can be for example double-stranded, partly double stranded, or single-stranded. Where single-stranded, the nucleic acid can be the sense strand or the antisense strand. The “nucleic acid” can be circular or linear. The term “nucleic acid” as used herein preferably encompasses DNA and RNA, specifically including genomic, hnRNA, pre-mRNA, mRNA, cDNA, recombinant or synthetic nucleic acids, including vectors.

By “encoding” is meant that a nucleic acid sequence or part(s) thereof corresponds, by virtue of the genetic code of an organism in question, to a particular amino acid sequence, e.g., the amino acid sequence of a desired polypeptide or protein. By means of example, nucleic acids “encoding” a particular polypeptide or protein, e.g. an enzyme, may encompass genomic, hnRNA, pre-mRNA, mRNA, cDNA, recombinant or synthetic nucleic acids.

Preferably, a nucleic acid encoding a particular polypeptide or protein may comprise an open reading frame (ORF) encoding said polypeptide or protein. An “open reading frame” or “ORF” refers to a succession of coding nucleotide triplets (codons) starting with a translation initiation codon and closing with a translation termination codon known per se, and not containing any internal in-frame translation termination codon, and potentially capable of encoding a polypeptide or protein. Hence, the term may be synonymous with “coding sequence” as used in the art.

The nucleic acids taught herein may encode more than one polypeptide or protein. Such nucleic acids are denoted as “polycistronic” nucleic acids and typically comprise several ORFs or coding sequences, each encoding a polypeptide or protein.

The terms “polypeptide” and “protein” are used interchangeably herein and generally refer to a polymer of amino acid residues linked by peptide bonds, and are not limited to a minimum length of the product. Thus, peptides, oligopeptides, polypeptides, dimers (hetero- and homo-), multimers (hetero- and homo-), and the like, are included within the definition. Both full-length proteins and fragments thereof are encompassed by the definition. The terms also include post-expression modifications of the polypeptide, for example, glycosylation, acetylation, phosphorylation, etc. Furthermore, for purposes of the present invention, the terms also refer to such when including modifications, such as deletions, additions and substitutions (e.g., conservative in nature), to the sequence of a native protein or polypeptide.

The term “biocatalyst” or “biochemical catalyst” generally refers to a substance, in particular an enzyme that initiates or modifies the rate of a biochemical reaction. The term “enzyme” as used herein denotes a molecule that catalyzes a chemical reaction. The term encompasses single enzymes, i.e. single catalytic entities, as well as systems comprising more than one catalytic entity. The enzymes described herein can naturally possess the recited activity or they can be engineered to exhibit said activity.

As used herein, “fatty acid enzyme” means any enzyme involved in fatty acid biosynthesis. As used herein, the term “fatty acid biosynthetic pathway” means a biosynthetic pathway that produces fatty acids. Fatty acid enzymes can be expressed or over-expressed in a host cell to produce fatty acids.

As used herein, the terms “purify,” “purified,” or “purification” means the removal or isolation of a molecule from its environment by, for example, isolation or separation. As used herein, these terms also refer to the removal of contaminants from a sample. For example, when α-olefins are produced in a host cell, the olefins can be purified by the removal of other cellular components (e.g., nucleic acids, polypeptides, lipids, carbohydrates, or other hydrocarbons). The terms “purify,” “purified,” and “purification” do not require absolute purity. They are relative terms.

As used herein, the terms “identity” and “identical” and the like refer to the sequence similarity between two polymeric molecules, e.g., between two nucleic acid molecules or polypeptides. Methods for comparing sequences and determining sequence identity are well known in the art. By means of example, percentage of sequence identity refers to a percentage of identical nucleic acids or amino acids between two sequences after alignment of these sequences. Alignments and percentages of identity can be performed and calculated with various different programs and algorithms known in the art. Preferred alignment algorithms include BLAST (Altschul, 1990; available for instance at the NCBI website) and Clustal (reviewed in Chenna, 2003; available for instance at the EBI website). Preferably, BLAST is used to calculate the percentage of identity between two sequences, such as the “Blast 2 sequences” algorithm described by Tatusova and Madden 1999 (FEMS Microbiol Lett 174: 247-250), for example using the published default settings or other suitable settings (such as, e.g., for the BLASTN algorithm: cost to open a gap=5, cost to extend a gap=2, penalty for a mismatch=−2, reward for a match=1, gap x_dropoff=50, expectation value=10.0, word size=28; or for the BLASTP algorithm: matrix=Blosum62, cost to open a gap=11, cost to extend a gap=1, expectation value=10.0, word size=3).

The term “renewable” is used herein to refer to a material (e.g. a molecule, a composition or a product) that can be produced or is derivable from a natural resource which is periodically (e.g., annually or perennially) replenished through the actions of plants of terrestrial, aquatic or oceanic ecosystems (e.g., agricultural crops, edible and non-edible grasses, forest products, seaweed, or algae), or microorganisms (e.g., bacteria, fungi, or yeast).

The term “renewable resource” refers to a natural resource that can be replenished within a 100 year time frame. The resource may be replenished naturally, or via agricultural techniques. Renewable resources include, for example but without limitation, plants, animals, fish, bacteria, fungi, yeasts, algae and forestry products. They may be naturally occurring, hybrids, or genetically engineered organisms. Natural resources such as crude oil, coal, and peat which take longer than 100 years to form are not considered to be renewable resources.

The term “bio-based content” refers herein to the amount of carbon from a renewable resource in a material as a percentage of the mass of the total organic carbon in the material, as determined by standard ASTM D6866.

The term “biosourced” with respect to a material (e.g. a molecule, a composition or a product) means that such material is derived from starting materials of renewable origin (i.e. from renewable resources). Accordingly, subject to typical measurement errors, a biosourced material has a bio-based content of at least 90%, preferably at least 95%, more preferably at least about 96%, 97% or 98%, even more preferably at least about 99% such as about 100%.

The present application generally relates to biocatalysts, in particular (free) fatty acid decarboxylases catalyzing the formation of α-olefins.

More particularly, the application provides novel P450 fatty acid decarboxylases that have improved (free) fatty acid decarboxylase activity compared to known P450 fatty acid decarboxylases, such as the decarboxylase isolated from the Staphylococcus massiliensis strain S46 (Sm46) as identified in WO2017001606. The application further provides nucleotide sequences encoding said novel P450 fatty acid decarboxylases, recombinant organisms comprising said nucleotide sequences, methods of production of α-olefins using said P450 fatty acid decarboxylases or said recombinant organisms and products obtained by these methods.

### Polypeptides

Disclosed herein are polypeptides having (free) fatty acid decarboxylase activity.

In particular, the present application relates to novel fatty acid decarboxylase enzymes that have high (free) fatty acid decarboxylase activity and for this reason are of interest for use in industrial processes. More particularly, the enzymes of the invention have improved (free) fatty acid decarboxylase activity compared to Sm46 or its truncated variant that has the N-terminal 29 amino acids deleted (Sm46Δ29, SEQ ID NO:6). In the context of the present invention, an “improved fatty acid decarboxylase” refers to an enzyme that has one or more of the following: a higher and/or faster (free) fatty acid substrate conversion, a higher and/or faster α-olefin production, and/or a higher ratio of decarboxylation activity over hydroxylation activity as compared to Sm46 or Sm46Δ29, more particularly as determined in an in vitro assay using one or more (free) fatty acid substrates as described herein.

The enzymes of the invention can be characterized by their amino acid sequence. More particularly, the polypeptides disclosed herein comprise an amino acid sequence having at least 85% such as at least 86%, 87%, 88% or 89% sequence identity to SEQ ID NO:6, wherein said amino acid sequence comprises the sequence of TLWHANTQRMESMDEVNIYRESIVL (SEQ ID NO: 7), in particular the sequence of TLWHANTQRMESMDEVNIYRESIVLLTKVGTRWAGVQAPPEDIERIATDMDIMIDSFRAL GGAFKGYKASKEARRRVEDWLEEQIIETRKGNIHPP (SEQ ID NO: 8), more particularly the sequence of TLWHANTQRMESMDEVNIYRESIVLLTKVGTRWAGVQAPPEDIERIATDMDIMIDSFRAL GGAFKGYKASKEARRRVEDWLEEQIIETRKGNIHPPEGTALYEFAHWEDYLG (SEQ ID NO: 9) or a sequence that is at least 90% or 95% such as at least 96%, 97%, 98% or 99% identical to SEQ ID NO:7, SEQ ID NO:8 or SEQ ID NO:9. In embodiments, the amino acid sequence comprises a sequence that is at least 90% identical to SEQ ID NO:7. In embodiments, the amino acid sequence comprises a sequence that is at least 95% identical to SEQ ID NO:7, SEQ ID NO:8 or SEQ ID NO:9. In particular embodiments, the amino acid sequence comprises a sequence that is identical to SEQ ID NO:7, SEQ ID NO:8 or SEQ ID NO:9.

Preferred polypeptides are those having an amino acid sequence comprising, consisting essentially of or consisting of SEQ ID NO:2 (P13G11) or SEQ ID NO:4 P3D3), and functional variants of these polypeptides; more preferably the polypeptides consisting of the amino acid sequence set forth in SEQ ID NO:2 (P13G11) or SEQ ID NO:4 (P3D3) and functional variants of these polypeptides.

The polypeptides disclosed herein have (free) fatty acid decarboxylase activity, i.e. they convert (free) fatty acids into α-olefins. The polypeptides disclosed herein have preferred (free) fatty acid decarboxylase activity, in that they preferably act on particular (free) fatty acids. More particularly, the novel decarboxylases described herein have preferred decarboxylase activity on C₁₀-C₁₆ (free) fatty acids, more particularly on even-numbered C₁₀-C₁₆ free fatty acids such as on C₁₀, C₁₂, C₁₄, and/or C₁₆ (free) fatty acids, even more particularly on C₁₀ or C₁₂ (free) fatty acids, i.e. the preferred substrate for these decarboxylase enzymes are C₁₀-C₁₆ free fatty acids, more particularly C₁₀ or C₁₂ free fatty acids.

Decarboxylase activity of a polypeptide can be assayed using routine methods. For example, the polypeptide can be contacted with a (free) fatty acid substrate, under conditions that allow the polypeptide to function. An increase in the level of an α-olefin can be measured to determine decarboxylase or olefin-producing activity.

As used herein in connection to a decarboxylase enzyme, a “preferred substrate” refers to the (free) fatty acid for which the polypeptide has the highest decarboxylase activity, i.e. when reacting with a number of (free) fatty acids, the polypeptide has the highest decarboxylase activity when the substrate is the preferred (free) fatty acid substrate. The substrate preference for a decarboxylase enzyme can be determined by calculating the conversion ratio of each (free) fatty acid substrate tested into corresponding α-olefin product as described elsewhere herein, wherein the preferred substrate is the (free) fatty acid substrate with the highest conversion ratio.

The polypeptides disclosed herein may further catalyze the hydroxylation of (free) fatty acids, in particular the α- and β-hydroxylation of (free) fatty acids, as side reactions, but the decarboxylation activity is the dominant activity of the polypeptides disclosed herein. In particular embodiments, the ratio of decarboxylation activity over hydroxylation activity is higher than 1.00 or 1.05, preferably higher than 1.10. The decarboxylase activity of a polypeptide can be determined by measuring the concentration of the corresponding α-olefin product when the polypeptide is reacted with a (free) fatty acid substrate, and the conversion ratio or conversion percentage of the (free) fatty acid substrate into the corresponding α-olefin product can be calculated as the ratio between the α-olefin concentration (e.g. in mM) and the initial (free) fatty acid concentration (e.g. in mM). Likewise, the hydroxylation activity of the polypeptide can be determined by measuring the concentration of the corresponding hydroxy fatty acid product(s) when the polypeptide is reacted with a (free) fatty acid substrate, and the conversion ratio or conversion percentage of the (free) fatty acid substrate into the corresponding hydroxy fatty acid product(s) can be calculated as the ratio between the hydroxy fatty acid concentration(s) (e.g. in mM) and the initial (free) fatty acid concentration (e.g. in mM).

In particular embodiments, the polypeptides described herein have a higher (free) fatty acid substrate conversion such as a higher C₁₀ or C₁₂ (free) fatty acid substrate conversion, as compared to Sm46 or Sm46Δ29. In further particular embodiments, the polypeptides have a higher C₁₂ (free) fatty acid substrate conversion as compared to Sm46 or Sm46Δ29. More particularly, the polypeptides described herein can convert at least 10% or 15%, preferably at least 20% or 25%, more preferably at least 30%, even more preferably at least 35% more C₁₂ (free) fatty acid substrate as compared to Sm46 or Sm46Δ29. In particular embodiments the (free) fatty acid decarboxylase activity is compared in vitro, using purified enzyme, (free) fatty acid substrate and analysis of the reaction products is performed e.g. by gas chromatography after extraction thereof. An exemplary method is described in the Examples section herein.

In particular embodiments, the polypeptides described herein have a higher α-olefin production, such as a higher C₉ or C₁₁ α-olefin production, as compared to Sm46 or Sm46Δ29. In further particular embodiments, the polypeptides have a higher C₁₁ α-olefin production as compared to Sm46 or Sm46Δ29. More particularly, the polypeptides described herein can produce at least 2.0 times such as at least 2.2, 2.4, 2.5, 2.6 or 2.8 times more, preferably at least 3 times more C₁₁ α-olefin production as compared to Sm46 or Sm46Δ29. In certain embodiments, the polypeptides described herein produce at least 20% more such as at least 21%, 22%, 23% or 24% more, preferably at least 25% more C₁₁ α-olefin.

Methods for quantifying (free) fatty acid substrate conversion and α-olefin production are well known to the skilled person. For instance, the (free) fatty acid substrate conversion can be quantified by calculating the (free) fatty acid substrate conversion ratio or percentage when the polypeptide is reacted with the (free) fatty acid substrate. The conversion ratio or conversion percentage for (free) fatty acid substrate can be calculated as the ratio between consumed substrate (e.g. difference between the initial (free) fatty acid concentration and the remaining (free) fatty acid concentration (e.g. in mM)) and the initial (free) fatty acid concentration (e.g. in mM). The α-olefin production can be determined based on the α-olefin concentration, or by calculating the conversion ratio or conversion percentage for the α-olefin. The conversion ratio or conversion percentage for an α-olefin can be calculated as the ratio between the α-olefin concentration (e.g. in mM) and the initial (free) fatty acid concentration (e.g. in mM). The α-olefin concentration and (free) fatty acid concentration in the reaction or culture medium can be measured by methods well known in the art, such as by GC/MS analysis.

In particular embodiments, the polypeptides described herein have at least about 80%, 85% or 90%, preferably at least about 95% such as at least about 96%, 97%, or 98%, more preferably at least about 99% sequence identity to SEQ ID NO:2 or SEQ ID NO:4 and have at least comparable fatty acid decarboxylase activity as the enzyme set forth in SEQ ID NO:2 (P13G11) or SEQ ID NO:4 (P3D3), i.e. have improved (free) fatty acid decarboxylase activity compared to Sm46 or its truncated variant that has the N-terminal 29 amino acids deleted (Sm46Δ29, SEQ ID NO:6).

Thus also envisaged herein are variant polypeptides of the polypeptides consisting of SEQ ID NO:2 (P13G11) or SEQ ID NO:4 (P3D3). Indeed, it will be understood by the skilled person that polypeptides with similar activity can be obtained by variant polypeptides characterized by conservative or non-essential amino acid substitutions of the novel polypeptides described herein, which do not have a substantial effect on the polypeptide functions. Whether or not a particular substitution will be tolerated (i.e., will not adversely affect desired biological properties) can be determined as described in Bowie et al. (1990) (Science 247:1306 1310). A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

Additional polypeptide variants are those in which additional amino acids are fused to the polypeptide, such as a secretion signal sequence, or a sequence which facilitates purification of the polypeptide.

Yet other polypeptide variants include functional or active fragments comprising at least about a consecutive stretch of amino acids corresponding to 80%, preferably 85% more preferably 90%, even more preferably 95% or more of P13G11 or P3D3, and which retain the same biological function as P13G11 or P3D3 (e.g. retain improved (free) fatty acid decarboxylase activity compared to Sm46 or its truncated variant that has the N-terminal 29 amino acids deleted (Sm46Δ29, SEQ ID NO:6)). Exemplary functional or active fragments include without limitation N- and/or C-terminally truncated forms of the polypeptides described herein, which retain the improved (free) fatty acid decarboxylase activity of P13G11 or P3D3. These functional or active fragments hence retain at least the decarboxylase catalytic domain of the polypeptide, i.e. the part of the polypeptide that is involved in the decarboxylase reaction.

Functional variants of the polypeptides described herein retain the decarboxylase activity of these polypeptides and may comprise or consist of an amino acid sequence having at least about 80%, 85% or 90%, preferably at least about 95% such as at least about 96%, 97%, or 98%, more preferably at least about 99% sequence identity in the decarboxylase catalytic domain of SEQ ID NO:2 or SEQ ID NO:4. The polypeptide variants may have an amino acid sequence substantially identical to SEQ ID NO:2 or SEQ ID NO:4 or they may have an amino acid sequence having at least about 80%, 85% or 90%, preferably at least about 95% such as at least about 96%, 97%, or 98%, more preferably at least about 99% sequence identity to SEQ ID NO:2 or SEQ ID NO:4.

The polypeptides envisaged herein can be produced by recombinant expression in a host cell. In particular embodiments, the polypeptide is secreted by the host cell. Alternatively, the polypeptide can be recovered from the host cell by cell lysis.

### Nucleic Acids

The application also provides recombinant nucleic acids encoding the novel P450 fatty acid decarboxylases described herein. These recombinant nucleic acids comprise at least the coding sequence for the polypeptides disclosed herein.

Nucleotide sequences encoding the polypeptides disclosed herein include the nucleotide sequences set forth in SEQ ID NO:1 (encoding P13G11) and SEQ ID NO:3 (encoding P3D3), as well as variants of these sequences.

Variant nucleotide sequences may for instance be codon-optimized sequences for recombinant expression in a host cell of choice. For instance, nucleotide sequences having at least about 80% or 85%, preferably at least 90%, 95%, 96%, 97% or 98%, more preferably at least about 99% sequence identity to SEQ ID NO:1 or SEQ ID NO:3 and encoding the polypeptides disclosed herein, are also envisaged herein.

Variants (or mutants) of the nucleotide sequences disclosed herein can be man-made e.g. using genetic engineering techniques. Such techniques are well known in the art and include, for example but without limitation, site directed mutagenesis, random chemical mutagenesis, and standard cloning techniques. Other exemplary techniques for mutagenesis are recombination techniques such as DNA shuffling that use fragments of existing sequences and mix them in novel combinations. The technique of DNA shuffling is well known in the art. Reference can be made to Stemmer (1994. Nature 370:389-391) for an exemplary shuffling technique.

Recombinant nucleic acids disclosed herein may further comprise regulatory sequences such as promoter and terminator sequences operatively linked to the coding sequence for the polypeptides disclosed herein.

Promoter and terminator sequences may be native to the host cell or exogenous to the host cell. Useful promoter and terminator sequences include those that are highly identical (i.e. having an identities score of 90% or more, preferably 95% or more, most preferably 99% or more) in their functional portions compared to the functional portions of promoter and terminator sequences, respectively, that are native to the host cell, particularly when the insertion of the recombinant nucleic acid is targeted at a specific site in the host genome. The use of native (to the host) promoters and terminators, together with their respective upstream and downstream flanking regions, can permit the targeted integration of the recombinant nucleic acid into specific loci of the host genome.

### Vector

Further disclosed herein are vectors that comprise a recombinant nucleic acid as described herein. In particular, a vector may comprise a coding sequence encoding an improved (free) fatty acid decarboxylase as described herein placed under the transcriptional control of one or more regulatory sequences such as one or more promoters and one or more terminators.

Numerous vectors are known to practitioners skilled in the art, and selection of an appropriate vector is a matter of choice.

The vectors can either be cut with particular restriction enzymes or used as circular DNA. The vector may contain restriction sites of various types for linearization or fragmentation.

Vectors may further contain a backbone portion (such as for propagation in E. coli) many of which are conveniently obtained from commercially available yeast or bacterial vectors.

The vector preferably comprises one or more selection marker gene cassettes. A “selection marker gene” is one that encodes a protein needed for the survival and/or growth of the transformed host in a selective culture medium. Typical selection marker genes encode proteins that (a) confer resistance to antibiotics or other toxins such as chloramphenicol, zeocin (sh ble gene from Streptoalloteichus hindustanus), genetecin, melibiase (MEL5), hygromycin (aminoglycoside antibiotic resistance gene from E. coli), ampicillin, tetracycline, or kanamycin (kanamycin resistance gene of Tn903), (b) complement auxotrophic deficiencies of the host. Two prominent examples of auxotrophic deficiencies are the amino acid leucine deficiency (e.g. LEU2 gene) or uracil deficiency (e.g. URA3 gene). Cells that are orotidine-5′-phosphate decarboxylase negative (ura3-) cannot grow on media lacking uracil. Thus a functional URA3 gene can be used as a marker on a host having a uracil deficiency, and successful transformants can be selected on a medium lacking uracil. Only host cells transformed with the functional URA3 gene are able to synthesize uracil and grow on such medium. If the wild-type strain does not have a uracil deficiency (as is the case with I. orientalis, for example), an auxotrophic mutant having the deficiency must be made in order to use URA3 as a selection marker for the strain. Methods for accomplishing this are well known in the art. The selection marker cassette typically further includes a promoter and terminator sequence, operatively linked to the selection marker gene, and which are operable in the host.

Successful transformants can be selected for in known manner, by taking advantage of the attributes contributed by the marker gene, or by other characteristics (such as ability to produce α-olefins) contributed by the inserted recombinant nucleic acids. Screening can also be performed by PCR or Southern analysis to confirm that the desired insertions, and optionally deletions have taken place, to confirm copy number and to identify the point of integration of coding sequences into the host genome. Activity (such as improved α-olefin-producing activity) of the polypeptide encoded by the inserted coding sequence can be confirmed using known assay methods as described elsewhere herein.

### Recombinant Host Cells

The application further provides genetically engineered host cells capable of producing α-olefins, in particular C₉-C₁₅ α-olefins, preferably C₉ or C₁₁ α-olefins, wherein said host cells are characterized in that they comprise a recombinant nucleic acid encoding an improved fatty acid decarboxylase as described hereinabove. In particular, the recombinant host cells comprise a recombinant nucleic acid comprising a nucleotide sequence that encodes a polypeptide that has at least about 80%, 85% or 90%, preferably at least about 95% such as at least about 96%, 97%, or 98%, more preferably at least about 99% sequence identity to SEQ ID NO:2 or SEQ ID NO:4 and maintains the activity of the enzyme set forth in SEQ ID NO:2 or SEQ ID NO:4 (i.e. retain improved (free) fatty acid decarboxylase activity compared to Sm46 or Sm46Δ29).

The genetically engineered host cells disclosed herein may be further genetically modified through expression of one or more recombinant nucleic acids encoding an enzyme involved in a fatty acid biosynthetic pathway. For example, the recombinant host cells may be further modified to overexpress a nucleic acid encoding an enzyme involved in an endogenous fatty acid biosynthetic pathway, or they may be further modified by introduction into the host cell of an exogenous nucleic acid encoding an enzyme involved in fatty acid synthesis. When expressed, the recombinant nucleic acid encoding the enzyme involved in fatty acid synthesis confers to the host cell the ability to produce or overproduce a fatty acid.

Each step within a fatty acid biosynthetic pathway can be modified to produce or overproduce a fatty acid of interest. For example, known genes involved in the synthesis of fatty acids can be expressed or overexpressed in a host cell to produce a desired free fatty acid, or attenuated to inhibit production of a non-desired fatty acid.

For instance, production of free fatty acids starting from acyl-ACP is ensured by thioesterases, whereafter the production of terminal olefins is catalyzed by a decarboxylase. Accordingly, exemplary genes involved in fatty acid synthesis include genes encoding a thioesterase.

In a preferred embodiment, the host cells are further genetically engineered to express or overexpress a thioesterase to induce or increase free fatty acid production. The chain length of a fatty acid substrate is controlled by the thioesterase, and hence, by (over)expressing a suitable thioesterase, a free fatty acid with desired carbon chain length can be obtained. Non-limiting examples of thioesterases are provided in Table 1. Preferably, thioesterases with (specific) activity on C₁₀ to C₁₆ acyl-ACP are used for (over)expression in the recombinant host cells described herein.

TABLE 1
Thioesterases

| GenBank accession | | | Preferential |
|---|---|---|---|
| number (UniProtKB/ | Source | | product |
| Swiss-Prot) | organism | Gene | produced |
| AAC73596 | E. coli | tesA without | C_{18:1} |
| | | leader sequence | |
| AAC73555 | E. coli | tesB | |
| AAA34215 | Umbellularia | fatB | C_{12:0} |
| (Q41635); | Californica | | |
| AAC49001 | | | |
| AAC49269 | Cuphea | fatB2 | C_{8:0}-C_{10:0} |
| (Q39513) | hookeriana | | |
| AAC49269; | Cuphea | fatB3 | C_{14:0}-C_{16:0} |
| AAC72881 | hookeriana | | |
| AAC49151 | Cinnamonum | fatB | C_{14:0} |
| (Q39473) | camphorum | | |
| CAA85388 | Arabidopsis | fatB | C_{16:1} |
| | thaliana | | |
| NP_189147; | Arabidopsis | fatA | C_{18:1} |
| NP_193041 | thaliana | | |
| CAC39106 | Bradyrhiizobium | fatA | C_{18:1} |
| | japonicum | | |
| AAC72883 | Cuphea | fatA | C_{18:1} |
| | hookeriana | | |
| AAL79361 | Helianthus annus | fatA1 | |
| JF338905 | Cocos nucifera | | C_{12:0} |

The thioesterase may be a thioesterase that is naturally present in higher plants. Two families of acyl-ACP thioesterases are present in higher plants: the “Class I” acyl-ACP thioesterases encoded by FatA genes, which are responsible for cleaving long-chain (for example, C₁₆ and C₁₈) unsaturated fatty acids from acyl-ACP, and the “Class 11” acyl-ACP thioesterases encoded by FatB genes, which are active on saturated fatty acyl chains, and which can be specific for medium-chain (C₈-C₁₄) acyl-ACPs or which can be active on both medium- and long-chain fatty acyl-ACPs. Non-limiting examples of thioesterases which are medium-chain fatty acid (MCFA)-specific and naturally present in plants are thioesterases encoded by FatB genes, or the thioesterases described for instance in Voelker et al. (1992 Science 257:72-74) and Jing et al. (2011 Biochemistry 12:44). The thioesterase may also be an engineered thioesterase as described for instance in Voelker et al. (1994 Journal of Bacteriology 176:7320-7327).

Depending on the α-olefin of interest, the expression of thioesterase enzymes may be either induced (by introduction into the host cell of an exogenous nucleic acid encoding said enzyme), stimulated (by overexpression of an endogenous gene encoding said enzyme) or attenuated (by modification of an endogenous gene encoding said enzyme).

In some situations, C₁₂ free fatty acids can be produced by expressing or overexpressing thioesterases that use C₁₂ acyl-ACP (for example, accession numbers Q41635 and JF338905) and attenuating thioesterases that produce non-C₁₂ fatty acids. In other instances, C₁₄ free fatty acids can be produced by attenuating endogenous thioesterases that produce non-C₁₄ fatty acids and (over)expressing the thioesterases that use C₁₄ acyl-ACP (for example, accession number Q39473).

Acetyl-CoA, malonyl-CoA, and free fatty acid overproduction can be verified using methods known in the art, for example, by using radioactive precursors, HPLC, and GC-MS subsequent to cell lysis.

For the production of C₁₁ α-olefins, the host cells are preferably modified by the introduction of an exogenous nucleic acid encoding a thioesterase having preferential hydrolase activity towards C₁₂ acyl-ACP substrate such as Q41635 or JF338905 and/or upregulating endogenous genes encoding a thioesterase having preferential hydrolase activity towards C₁₂ acyl-ACP, and optionally downregulating endogenous genes encoding thioesterases that produce non-C₁₂ fatty acids.

The free fatty acids produced by the fatty acid enzymes in the host cells envisaged herein are preferably substrates of the improved (free) fatty acid decarboxylase enzymes described herein.

Accordingly, particularly preferred host cells for the production of C₉-C₁₅ α-olefins are recombinant host cells comprising:
a recombinant nucleic acid encoding a thioesterase having preferential hydrolase activity towards C₁₀ to C₁₆ acyl-ACP substrate; and
a recombinant nucleic acid encoding an improved fatty acid decarboxylase as described herein.

Particularly preferred host cells for the production of C₁₁ α-olefins are recombinant host cells comprising:
a recombinant nucleic acid encoding a thioesterase having preferential hydrolase activity towards C₁₂ acyl-ACP substrate; and
a recombinant nucleic acid encoding an improved fatty acid decarboxylase as described herein.

Particularly preferred host cells for the production of C₉ α-olefins are recombinant host cells comprising:
a recombinant nucleic acid encoding a thioesterase having preferential hydrolase activity towards C₁₀ acyl-ACP substrate; and
a recombinant nucleic acid encoding an improved fatty acid decarboxylase as described herein.

In particular embodiments, where the above nucleotide sequences encoding a thioesterase are endogenously expressed by the host cell, it is envisaged that expression of these sequences can be increased specifically so as to ensure commercially relevant α-olefin production.

More particularly, in particular embodiments, the host cell is selected to have a high endogenous thioesterase activity. Methods of selecting cells having particular properties are known in the art.

Any cell that can be suitably transformed with and/or genetically engineered to ensure (over)expression of one or more of the described recombinant nucleic acids can be used in the context of the present invention. The host cells disclosed herein can be any prokaryotic or eukaryotic organism or cell. Non-limiting examples of host cells include plant cells, bacterial cells, yeast cells, fungal cells, and algal cells. In embodiments, the host cells are genetically engineered bacteria, or genetically engineered fungi, in particular yeasts, genetically engineered algae, or genetically engineered plant cells.

Preferably, the host cells are oleaginous host cells. For example, the host cell may be an oleaginous bacterium, an oleaginous fungus, oleaginous yeast or an oleaginous alga. Non-limiting examples of oleaginous yeasts include Lipomyces starkeyi, Rhodosporidium toruloides, Rhodotorula glutinis, and Yarrowia lipolytica. Non-limiting examples of oleaginous algae genera include Botryococcus, Chaetoceros, Chlorella, Chlorococcum, Cylindrotheca, Dunaliella, Fistulifera, Isochrysis, Nannochloropsis, Neochloris, Nitzschia, Pavlova, Scenedesmus, Skeletonema, Stichococcus and Tetraselmis.

Thus, the genetically engineered host cells disclosed herein comprise a recombinant nucleic acid encoding an improved fatty acid decarboxylase disclosed herein, and optionally one or more recombinant nucleic acids encoding a fatty acid enzyme, i.e. an enzyme involved in fatty acid synthesis. Additionally or alternatively the expression of one or more genes encoding enzymes involved in the production of fatty acids other than C₁₀-C₁₆ free fatty acids may be suppressed, decreased or limited.

The methods for generating the genetically engineered host cells described herein involve standard genetic modifications, for which well-established methods are available to the skilled person.

Genetic engineering of the host cells to contain a recombinant nucleic acid encoding a an improved fatty acid decarboxylase or a fatty acid enzyme as described herein is accomplished in one or more steps via the design and construction of appropriate vectors and transformation of the host cells with those vectors.

Electroporation and/or chemical (such as calcium chloride- or lithium acetate-based) transformation methods or Agrobacterium tumefaciens-mediated transformation methods as known in the art can be used.

Also disclosed herein are methods for obtaining a genetically engineered host cell capable of producing an α-olefin of interest as described herein, which method may comprise transforming a host cell with a recombinant nucleic acid encoding an improved fatty acid decarboxylase as taught herein and optionally one or more recombinant nucleic acids encoding a fatty acid enzyme as taught herein, more particularly a fatty acid enzyme involved in the synthesis of a substrate for the improved fatty acid decarboxylase as taught herein. In particular, the method may comprise the steps of:
a) transforming a host cell with a recombinant nucleic acid encoding an improved fatty acid decarboxylase as taught herein and optionally one or more recombinant nucleic acids encoding a fatty acid enzyme as taught herein; and
b) selecting a host cell capable of producing an α-olefin of interest.

In particular embodiments, the method further comprises modifying said host cell so as to reduce the endogenous production of olefins other than the α-olefin of interest.

As detailed above, different genetic modifications are envisaged herein which induce α-olefin production, in particular C₉-C₁₅ α-olefin production, in a host cell. Accordingly, the present invention also relates to the use of the genetically engineered host cells as described herein for the production of α-olefins, more particularly C₉-C₁₅ α-olefins.

### Producing α-Olefins Using Recombinant Host Cells

In a further aspect, methods are provided for the production of C₉-C₁₅ α-olefins, more particularly C₉ or C₁₁ α-olefins, which method comprises providing a genetically engineered host cell as described above and culturing said genetically engineered host cell in a culture medium so as to allow the production of C₉-C₁₅ α-olefins. More particularly, the host cell is cultured under conditions suitable to ensure expression or overexpression of the improved (free) fatty acid decarboxylase disclosed herein and optionally one or more fatty acid enzyme(s) involved in the synthesis of the substrate of the decarboxylase.

In particular embodiments, the host cells ensure a rate of α-olefin production, more particularly C₉-C₁₅ α-olefin production, which is sufficiently high to be industrially valuable. Indeed, the recombinant host cells disclosed herein may be capable of ensuring a high yield at limited production costs. Furthermore, they are capable of producing α-olefins of desired carbon chain length. Indeed, the decarboxylase enzymes envisaged herein preferably have substrate preference for C₁₀-C₁₆ free fatty acids, more particularly C₁₀ or C₁₂ free fatty acid. Also advantageously, the production of unwanted co-products such as hydroxyl fatty acids is minimal. Indeed, the polypeptides disclosed herein have specific decarboxylase activity. Moreover, the improved (free) fatty acid decarboxylases disclosed herein were shown to have increased catalytic rates for α-olefin production, particularly at low enzyme concentrations. This allows the use of less enzyme for the same α-olefin production, which results in a higher benefit-cost ratio.

The recombinant host cells are cultured under conditions suitable for the production of C₉-C₁₅ α-olefins by the host cells. More particularly this implies “conditions sufficient to allow (over)expression” of the recombinant nucleic acid as described herein, which means any condition that allows a host cell to (over)produce an improved fatty acid decarboxylase or a fatty acid enzyme as described herein. Suitable conditions include, for example, fermentation conditions. Fermentation conditions can comprise many parameters, such as temperature ranges, levels of aeration, and media composition.

Each of these conditions, individually and in combination, allows the host cell to grow. To determine if conditions are sufficient to allow (over)expression, a host cell can be cultured, for example, for about 4, 8, 12, 18, 24, 36, or 48 hours. During and/or after culturing, samples can be obtained and analyzed to determine if the conditions allow (over)expression. For example, the host cells in the sample or the culture medium in which the host cells were grown can be tested for the presence of a desired product. When testing for the presence of a desired product, assays, such as, but not limited to, sodium dodecyl sulfate polyacrylamide gel electrophoresis (SDS-PAGE), TLC, HPLC, GC/FID, GC/MS, LC/MS, MS, can be used.

Exemplary culture media include broths or gels. Micro-organisms are typically grown in a culture medium comprising a carbon source to be used for growth of the micro-organism. Exemplary carbon sources include carbohydrates, such as glucose, fructose, cellulose, or the like, that can be directly metabolized by a micro-organism. In addition, enzymes can be added to the culture medium to facilitate the mobilization (e.g., the depolymerization of starch or cellulose to fermentable sugars) and subsequent metabolism of the carbon source. A culture medium may optionally contain further nutrients as required by the particular strain, including inorganic nitrogen sources such as ammonia or ammonium salts, and the like, and minerals and the like.

Other growth conditions, such as temperature, cell density, and the like are generally selected to provide an economical process. Temperatures during each of the growth phase and the production phase may range from above the freezing temperature of the medium to about 50° C.

The culturing step of the methods described herein may be conducted aerobically, anaerobically, or substantially anaerobically. Briefly, anaerobic conditions refer to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen.

The percent of oxygen can be maintained by, for example, sparging the culture with an N₂/CO₂ mixture or other suitable non-oxygen gas or gasses.

The cultivation step of the methods described herein can be conducted continuously, batch-wise, or some combination thereof.

In particular embodiments, wherein phototrophic algae are used as host cells, the method for the production of α-olefins may comprise providing algae genetically engineered to (over)produce α-olefins as taught herein, and culturing said algae in photobioreactors or an open pond system using CO₂ and sunlight as feedstock.

In certain embodiments, the conditions suitable for the production α-olefins may further imply cultivating the host cells in a culture medium which comprises at least one fatty acid substrate, which is converted into the corresponding α-olefin by the decarboxylase encoded by the recombinant nucleic acid comprised in the host cell.

Preferably, the fatty acid substrate is a saturated free fatty acid substrate. Also preferably, the fatty acid substrate is a straight chain free fatty acid substrate. Also preferably, the fatty acid substrate is an even-numbered C₁₀-C₁₆ free fatty acid substrate (i.e. a free C₁₀, C₁₂, C₁₄ or C₁₆ free fatty acid substrate or any combination thereof), more preferably a C₁₀ or C₁₂ free fatty acid substrate.

Particularly intended herein is the production of C₉-C₁₅ α-olefins, more particularly C₉ or C₁₁ α-olefins. C₉-C₁₅ α-olefins, more particularly C₉ or C₁₁ α-olefins can be obtained using a recombinant host cell described herein specifically modified for the production of C₉-C₁₅ α-olefins, more particularly C₉ or C₁₁ α-olefins.

In further embodiments, methods are provided for producing C₉-C₁₅ α-olefins, more particularly C₉ or C₁₁ α-olefins, which, in addition to the steps detailed above, further comprise the step of recovering the α-olefins from the host cell or the culture medium. Suitable purification can be carried out by methods known to the person skilled in the art such as by using lysis methods, extraction, ion exchange resins, electrodialysis, nanofiltration, etc.

Accordingly, methods are provided for the production of C₉-C₁₅ α-olefins, more particularly C₉ or C₁₁ α-olefins, which methods comprise the steps of:

(i) providing a genetically engineered host cell as described herein;
(ii) culturing the host cells in a culture medium under conditions suitable for the production of C₉-C₁₅ α-olefins, and
(iii) recovering the α-olefins from the host cell or the culture medium.

In particular embodiments, wherein oleaginous yeasts are used as host cells, the method for the production of C₉-C₁₅ α-olefins may comprise the following steps:

(i) providing oleaginous yeasts genetically engineered to (over)produce C₉-C₁₅ α-olefins as taught herein;
(ii) culturing said oleaginous yeasts in fermenters; and
(iii) recovering the α-olefins from the oleaginous yeast or the culture medium.

In particular embodiments, wherein phototrophic algae are used as host cells, the method for the production of C₉-C₁₅ α-olefins may comprise the following steps:

(i) providing algae genetically engineered to (over)produce C₉-C₁₅ α-olefins as taught herein;
(ii) culturing said algae in photobioreactors or an open pond system using CO₂ and sunlight as feedstock; and
(iii) recovering the α-olefins from the algae or the culture medium.

In particular embodiments the host cells are cultivated under conditions which allow secretion of α-olefins into the environment.

Typically, in the methods for the production of C₉-C₁₅ α-olefins envisaged herein, the decarboxylase expressed by the host cell is not secreted by said host cell and the α-olefin is produced inside the host cell. However, in particular embodiments or for particular applications, it is of interest to ensure secretion of the decarboxylase by the host cells provided herein. This can be of interest where the enzyme is envisaged to be active upon secretion into its environment. A secretion signal sequence can be operably linked to the nucleic acid encoding the improved (free) fatty acid decarboxylase to this end. In this connection, “operably linked” denotes that the sequence encoding the secretion signal peptide and the sequence encoding the polypeptide to be secreted are connected in frame or in phase, such that upon expression the signal peptide facilitates the secretion of the polypeptide so-linked thereto.

### Production of α-Olefins Using Polypeptides

Also provided herein is the use of the improved (free) fatty acid decarboxylase enzymes disclosed herein for the production of C₉-C₁₅ α-olefins, more particularly C₉ or C₁₁ α-olefins.

Some methods described herein relate to the production of C₉-C₁₅ α-olefins, more particularly C₉ or C₁₁ α-olefins, using a (purified) improved (free)fatty acid decarboxylase enzyme disclosed herein and a (free) fatty acid substrate. Accordingly, disclosed herein is a method for the production of C₉-C₁₅ α-olefins, more particularly C₉ or C₁₁ α-olefins, comprising contacting an improved (free) fatty acid decarboxylase as disclosed herein with a suitable free fatty acid substrate so as to produce C₉-C₁₅ α-olefins, more particularly C₉ or C₁₁ α-olefins.

For example, a host cell can be genetically engineered to overexpress an improved (free) fatty acid decarboxylase as disclosed herein. The recombinant host cell can be cultured under conditions sufficient to allow (over)expression of the decarboxylase. Cell-free extracts can then be generated using known methods. For example, the host cells can be lysed using detergents or by sonication. The overexpressed polypeptides can be purified using known methods, or the cell-free extracts can be used as such for the production of α-olefins. The host cells can also be genetically engineered to (over)express an improved (free) fatty acid decarboxylase as disclosed herein and to secrete said polypeptide into the growth medium as described elsewhere. The secreted polypeptides can then be separated from the growth medium and optionally purified using known methods without the need for obtaining cell-free extracts.

Next, (free) fatty acid substrates can be added to the cell-free extracts or (purified) enzymes and maintained under conditions to allow conversion of the (free) fatty acid substrates to α-olefins. The α-olefins can then be separated and purified using known techniques.

Olefins having particular branching patterns, levels of saturation, and carbon chain length can be produced from free fatty acid substrates having those particular characteristics using the described methods. For example, the fatty acid substrate may be an unsaturated free fatty acid substrate (e.g. a monounsaturated free fatty acid substrate), or a saturated free fatty acid substrate. The fatty acid substrate may be a straight chain free fatty acid substrate, a branched chain free fatty acid substrate, or a free fatty acid substrate that includes a cyclic moiety.

Preferably, the fatty acid substrate is a saturated free fatty acid substrate. Also preferably, the fatty acid substrate is a straight chain free fatty acid substrate. Also preferably, the fatty acid substrate is an even-numbered C₁₀-C₁₆ free fatty acid substrate (i.e. a C₁₀, C₁₂, C₁₄ and/or C₁₆ free fatty acid substrate), more preferably a C₁₂ free fatty acid substrate.

Particularly intended herein is the production of C₁₁ α-olefins. C₁₁ α-olefins can be obtained from a C₁₂ free fatty acid substrate using an enzyme having C₁₂ free fatty acid decarboxylase activity. In particular embodiments, a method is provided for the production of C₁₁ α-olefins, which method comprises contacting a polypeptide of the invention with a C₁₂ free fatty acid substrate, preferably dodecanoic acid (or lauric acid).

Also particularly intended herein is the production of C₉ α-olefins. C₉ α-olefins can be obtained from a C₁₀ free fatty acid substrate using an enzyme having C₁₀ free fatty acid decarboxylase activity. In particular embodiments, a method is provided for the production of C₉ α-olefins, which method comprises contacting a polypeptide of the invention with a C₁₀ free fatty acid substrate.

### α-Olefins

Also provided herein are C₉-C₁₅ α-olefins, more particularly C₉ or C₁₁ α-olefins, and compositions comprising C₉-C₁₅ α-olefins, more particularly C₉ or C₁₁ α-olefins, obtainable by the methods disclosed herein.

The methods described herein advantageously result in the production of homogenous α-olefins, wherein the α-olefins produced have a uniform carbon chain length. These processes are hence more efficient than conventional processes which result in the production of mixture of α-olefins with different carbon chain length and which require separation of the different α-olefins for subsequent reactions.

### Post-Production Processing

The produced α-olefins, more particularly C₉-C₁₅ α-olefins such as C₉ or C₁₁ α-olefins, can be used as or converted into a fuel, in particular a biofuel. These α-olefins, more particularly C₉-C₁₅ α-olefins such as C₉ or C₁₁ α-olefins, can also be used as starting material for the production of chemicals or personal care additives (e.g. polymers, surfactants, plastics, textiles, solvents, adhesives, etc.). They can also be used as feedstock for subsequent reactions, such as hydrogenation and/or oligomerization reactions, to make other products.

A further aspect of the invention relates to a method for the production of poly-α-olefins (PAO), said method comprising:
a) producing α-olefins, more particularly C₉-C₁₅ α-olefins, according to a method disclosed herein;
b) oligomerizing the α-olefins produced in step a); and optionally
c) hydrogenating the oligomer produced in step b).

In particular embodiments, a method is provided for the production of C₃₃ PAOs, which comprises:
a) producing C₁₁ α-olefins according to a method disclosed herein;
b) trimerizing the C₁₁ α-olefins produced in step a); and optionally
c) hydrogenating the trimer produced in step b).

In particular embodiments, a method is provided for the production of C₂₇ PAOs, which comprises:
a) producing C₉ α-olefins according to a method disclosed herein;
b) trimerizing the C₉ α-olefins produced in step a); and optionally
c) hydrogenating the trimer produced in step b).

Oligomerization of C₉-C₁₅ α-olefins in the presence of a catalyst is well known in the art. Catalysts that can be used for the oligomerization step are for example, but not limited to, AlCl₃, BF₃, BF₃ complexes for cationic oligomerization, and metal based catalysts like metallocenes.

Following the oligomerization step, residual unsaturation that is potentially present in the oligomers is saturated by catalytic hydrogenation resulting in saturated aliphatic hydrocarbons with one or more side branches.

The oligomers obtained by methods as described herein are known under the generic name of poly-α-olefins (PAO). The PAO production methods described herein advantageously result in the homogenous production of PAOs of a well-defined carbon chain length. Accordingly, the application also provides a composition of PAO's obtainable by a PAO production method described herein, characterized in that at least 50%, preferably at least 85% or 90%, more preferably at least 95% such as 96%, 97%, 98% or even 99% of the PAOs have a well-defined carbon chain length such as C₂₇ or C₃₃ PAOs.

The methods provided herein allow obtaining a base oil with a well-defined viscosity. The PAOs, more particularly the C₂₇ or C₃₃ PAOs, obtainable by a method as described herein can be used as base oils, which display very attractive viscosity indices, with the viscosity increasing with the number of carbons. These base oils can be used, together with additives and optionally other base oils, to formulate lubricants. In particular, PAOs with a number of carbons of about 30, more particularly 33 carbons, are preferred for automotive lubricants.

Accordingly, a further aspect relates to the use of the improved (free) fatty acid decarboxylase enzymes disclosed herein and the recombinant host cells described herein for the industrial production of lubricants.

Also provided herein are lubricants comprising poly-α-olefins, more particularly lubricants comprising poly-α-olefins which contain a more homogenous composition of poly-α-olefins, more particularly a high concentration of poly-olefins of a well-defined length, such as those obtainable by a method as described herein. Indeed, the methods disclosed herein allows for the production of lubricants which are produced based on biosourced C₉-C₁₅ α-olefins. More particularly, the invention allows for the provision of lubricants comprising poly-α-olefins, whereby at least 50%, preferably at least 85% or 90%, more preferably at least 95% such as 96%, 97%, 98% or even 99% of said poly-α-olefins are poly-α-olefins of a well defined carbon chain length, such as C₂₇ or C₃₃ poly-α-olefins.

Further disclosed herein are methods for the production of alkanes, said methods comprising: (a) production of α-olefins more particularly C₉-C₁₅ α-olefins, according to a method disclosed herein; and (b) hydrogenation of the α-olefins obtained in step (a) to produce alkanes.

The present invention will now be further illustrated by means of the following non-limiting examples.

### EXAMPLES

### Example 1: Production of C₁₁ α-Olefins by the Novel Fatty Acid Decarboxylase of the Invention

### Material and Methods

### Molecular Cloning

The coding sequences of P26H9 (amino acid sequence set forth in SEQ ID NO:10), P40E6 (amino acid sequence set forth in SEQ ID NO:11), P42E11 (amino acid sequence set forth in SEQ ID NO:12), P21G12 (amino acid sequence set forth in SEQ ID NO:13), P41A3 (amino acid sequence set forth in SEQ ID NO:14) and P13G11 (SEQ ID NO:1) were cloned into the NdeI/XhoI sites of the pET28b plasmid (Novagen) by standard molecular biology techniques.

### Protein Overexpression and Purification

E. coli BL21(DE3) cells (Novagen) carrying a recombinant plasmid or an empty PET28b plasmid (control) were cultured for several cycles to ensure best growth state at 37° C. in 10 mL LB medium supplemented with 50 μg/ml of kanamycin, followed by inoculation (1:100 ratio) into 1 L fresh Terrific Broth medium containing 50 μg/ml of kanamycin, 1 mM thiamine, 4% glycerol and a rare salt solution (6750 mg/l FeCl₃, 500 mg/l ZnCl₂, 500 mg/l CoCl₂, 500 mg/l Na₂MoO₄, 250 mg/l CaCl₂, 465 mg/l CuSO₄, and 125 mg/l H₃BO₃) at 37° C. Cells were grown at 37° C. for 3 to 4 h until the optical density at 600 nm (OD₆₀₀) reached 0.6 to 0.8, at which 0.2 mM isopropyl-β-_{D}-thiogalactopyranoside (IPTG) and 0.5 mM δ-aminolevulinic acid were added, followed by 24 h of cultivation at 16° C. with shaking at 150 rpm.

Then, the cell cultures were recovered by centrifugation at 6000 rpm, 4° C. The cell pellets were re-suspended in 40 mL lysis buffer (50 mM NaH₂PO₄, 300 mM NaCl, 10% glycerol, and 10 mM imidazole, pH 8.0). The re-suspended cells were disrupted by ultrasonication and the cell lysates were clarified by centrifugation at 13,000×g for 30 min (4° C.) to remove cell debris. Purification of the His-tagged proteins was carried out as described by Liu et al. (2014. Biotechnology for Biofuels 7:28) with minor modifications. Briefly, to the clarified cell lysate, 1 mL of Ni-NTA resin was added and gently mix incubated at 4° C. for 1 h. The slurry was then loaded onto an empty column and washed with approximately 100 mL of wash buffer (50 mM NaH₂PO₄, 300 mM NaCl, 10% glycerol, and 20 mM imidazole, pH 8.0) until no proteins were detectable in flowthrough. The bound target proteins were eluted with elution buffer (50 mM NaH₂PO₄, 300 mM NaCl, 10% glycerol, and 250 mM imidazole, pH 8.0). The eluates were pooled and concentrated with an Amicon Ultra centrifugal filter (30 kDa cutoff). Imidazole contained in the protein eluates was removed by ultrafiltration and buffer exchange on a PD-10 column into storage buffer (50 mM NaH₂PO₄, 300 mM NaCl, 10% glycerol, pH 7.4). The final purified protein was flash-frozen with liquid nitrogen and stored at −80° C. for later use.

### In Vitro Enzymatic Assays

In vitro olefin biosynthesis was evaluated by adding to 0.5 μM of the purified enzyme, 200 μM C₁₂ fatty acid substrate (TCI (Shanghai) Development Co., Ltd.n solubilized in DMSO at 20 mM stock solution) and 220 μM H₂O₂ in a total volume of 200 μl. The reaction mixture was incubated at 30° C. for 40 min. Reactions were quenched by the addition of 20 μl of 10 M HCl. The reaction mixture was extracted by 200 μl ethyl acetate.

Following extraction, the organic phase was collected and analyzed by gas chromatography as described below.

### Gas Chromatography (GC)

The GC analytical method for hydrocarbon and fatty acid samples was adapted from Guan et al. (2011 J Chromatogr A 1218:8289-8293). The analyses were performed on an Agilent 7890B gas chromatograph equipped with a capillary column HP-INNOWAX (Agilent Technologies, Santa Clara, Calif., USA; cross-linked polyethylene glycerol, i.d. 0.25 μm film thickness, 30 m by 0.25 mm). The helium flow rate was set to 1 ml per minute. The oven temperature was controlled initially at 40° C. for 4 min, then increased at the rate of 10° C. per min to 250° C., and held for 5 min. The injecting temperature was set to 280° C. with the injection volume of 1 μl under splitless injection conditions. The response factors between fatty acids and alkenes were determined by analyzing known authentic fatty acids (C₁₀-C₂₀), 1-alkenes (C₉-C₁₉) and 1-heptadecanoic acid standards as described in Liu et al. (2014 Biotechnol. Biofuels 7:28).

### Results

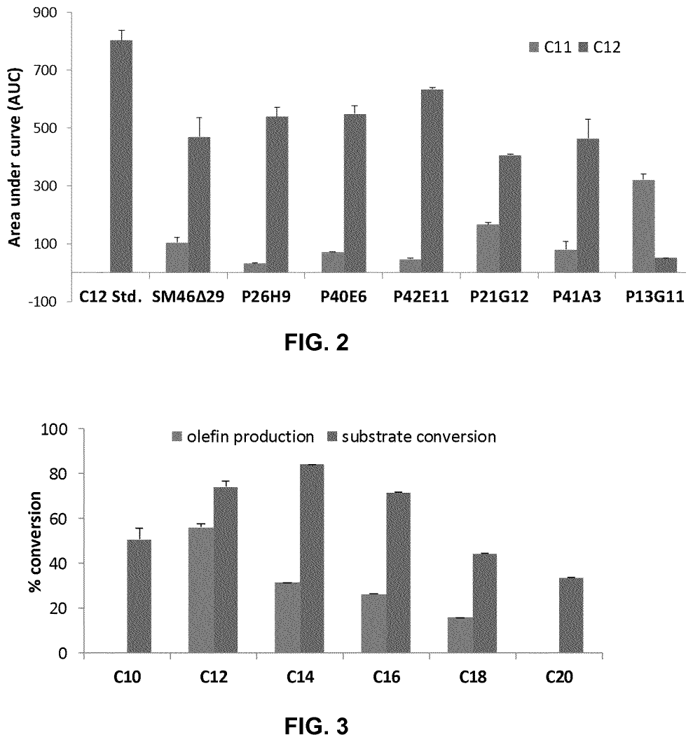

FIG. 2 shows C₁₂ lauric acid conversion and C₁₁ α-undecene production for all tested enzymes: Sm46Δ29 and the novel decarboxylase enzymes of the invention. P13G11 exhibited the highest conversion and olefin production from the C₁₂ fatty acid substrate (FIG. 2).

### Example 2: Substrate Preference of P13G11

### Material and Methods

The recombinantly expressed and purified P13G11 from Example 1 was incubated with different fatty acid substrates as described in Example 1 to assess the substrate preference of the P13G11 enzyme. Briefly, 200 μl reaction mixtures containing 200 μM of each fatty acid substrate, 220 μM H₂O₂ and 2 μM of the purified P13G11 enzyme were incubated at 30° C. for 2 hours. Reactions were quenched by the addition of 20 μl of 10 M HCl. The reaction mixture was extracted by 200 μl ethyl acetate. Following extraction, the organic phase was collected and analyzed by gas chromatography as described in Example 1.

### Results

FIG. 3 shows that the C₁₂ fatty acid is the best olefin-producing substrate for P13G11.

### Example 3: Fatty Acid Decarboxylation and Hydroxylation Activities of Sm46Δ29 and P13G11 at Different Enzyme Concentrations

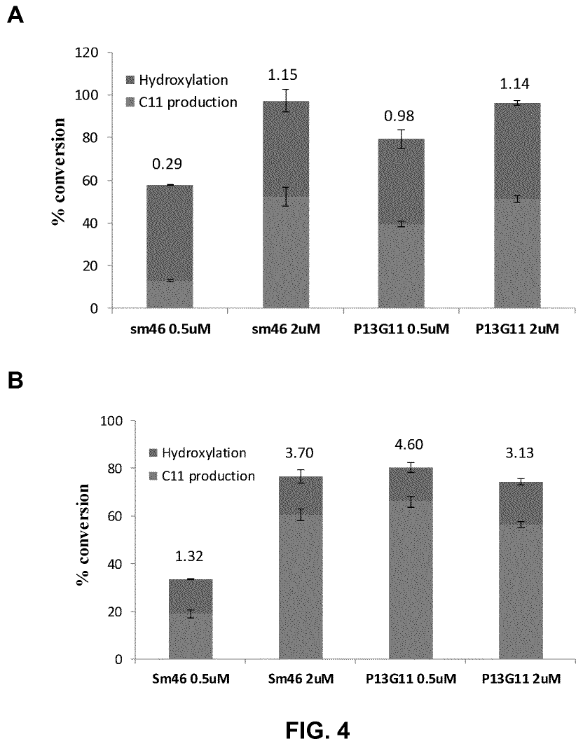

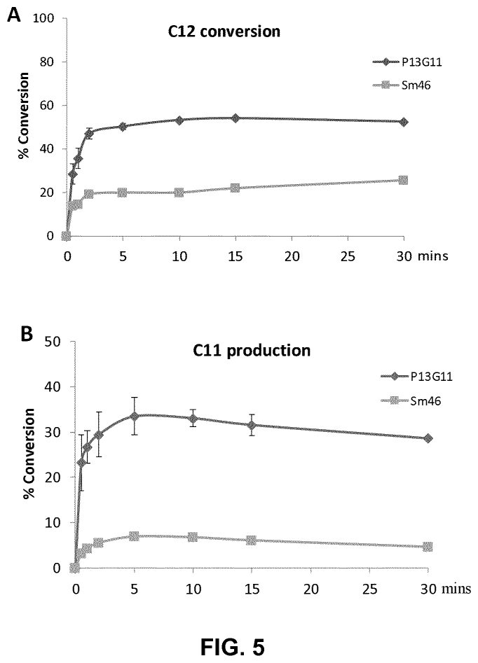

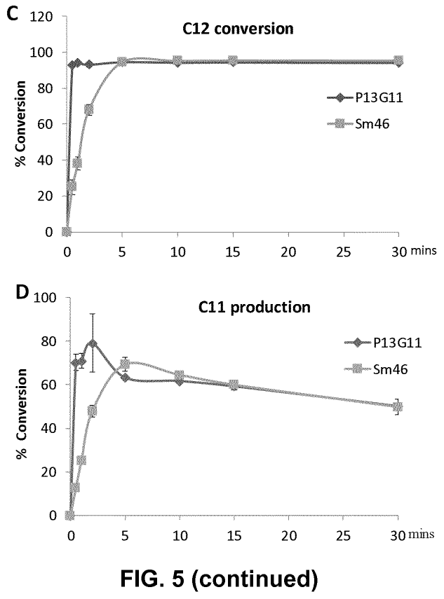

Recombinantly expressed and purified Sm46Δ29 and P13G11 from Example 1 were reacted with C₁₂ fatty acid substrate, and the decarboxylation versus hydroxylation activity of the enzyme P13G11 was compared with its wild type parent Sm46Δ29 at different enzyme concentrations and for different incubation times (FIG. 4). In addition, the C₁₂ fatty acid substrate conversion and olefin production ratios were plotted against time to compare the reaction rates of the enzymes (FIG. 5).

### Materials and Methods

### In Vitro Enzymatic Assays

2 ml NaPO₄ buffer (pH 7.4) containing 200 μM C₁₂ fatty acid substrate, 220 μM H₂O₂ and 0.5 μM or 2 μM of the purified enzymes were incubated at 30° C. At t=0, 0.5, 1, 2, 5, 10, 15, and 30 min, 200 μl samples were taken from the reaction mixture for studying the reaction rates of the enzymes. At t=40 min and 2 h, 200 μl samples were taken from the reaction mixture for studying hydroxylation versus decarboxylation activity of the enzymes. The samples were extracted by 200 μl ethyl acetate.

### Analysis of Decarboxylase Activity

Following extraction, the organic phase was collected and analyzed by gas chromatography as described in Example 1 for analyzing decarboxylase activity.

### Analysis of Hydroxylation Activity

The hydroxylation activity was estimated by subtracting the alkene production from the total substrate conversion. This indirect, but more convenient, method was validated with C₁₄ myristic acid substrate by direct measurement of the BSTFA/TMCS derivatized hydroxylation products (Xu et al. 2017 Biotechnology for Biofuels 10:208).

### Results

It was revealed that the activity improvement of P13G11 over Sm46Δ29 looked more prominent when at the low enzyme concentration of 0.5 μM than that of at the saturating enzyme concentration of 2.0 μM, indicating a faster catalytic rate and/or a better stability against H₂O₂. In addition, the decarboxylation product ratio is generally higher when the reactions were incubated for an extended time (FIG. 4).

P13G11 exhibited a significantly improved rate of C₁₂ fatty acid substrate conversion compared to Sm46Δ29 (FIGS. 5A&C). The slightly dropped olefin production ratio in FIGS. 5B&D at the longer incubation time points may reflect the loss caused by the semi-volatility of the C₁₁ alkene after repeated sampling at the early time points from the same test tubes.

### Example 4: Fatty Acid Decarboxylation Activity of P3D3

### Materials and Methods

The coding sequence of P3D3 (SEQ ID NO:3) was cloned into the pET28b plasmid, and recombinantly expressed in E. coli BL21(DE3) cells as described in Example 1. Purification of the P3D3 enzyme was carried out as described in Example 1. 2 ml NaPO₄ buffer (pH 7.4) containing 200 μM C₁₂ fatty acid substrate, 220 μM H₂O₂ and 0.5 μM of the purified P3D enzyme or of P13G11 of Example 1 were incubated at 30° C. At t=0, 0.5, 1, 2, 5, 10, 15 and 30 min, 200 μl samples were taken from the reaction mixture for analysis. The samples were extracted by 200 μl ethyl acetate. Following extraction, the organic phase was collected and analyzed by gas chromatography as described in Example 1.

### Results

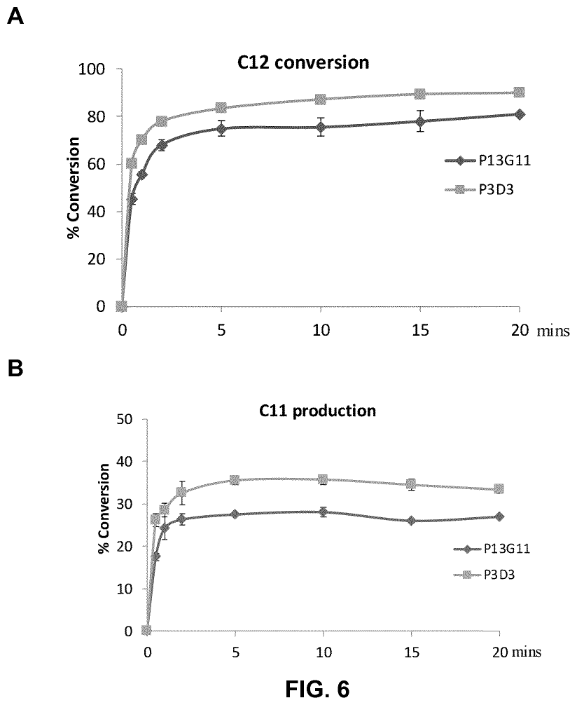

P3D3 also showed improved decarboxylation activity against the mid-chain length fatty acids, in particular C₁₂ fatty acid. The time course analysis of the fatty acid decarboxylation activity of this decarboxylase verified its further improved catalytic reaction rate (FIG. 6).

1. A polypeptide having fatty acid decarboxylase activity, wherein said polypeptide comprises an amino acid sequence having at least 95%, preferably at least 99%, identity to SEQ ID NO:4 or SEQ ID NO:2.

2. The polypeptide according to claim 1, wherein said polypeptide converts at least 2.0 times more C₁₂ free fatty acids to C₁₁ α-olefins as compared to the enzyme set forth in SEQ ID NO:6.

3. The polypeptide according to claim 1, wherein said polypeptide converts at least 10% more C₁₂ (free) fatty acid substrate in vitro as compared to the enzyme set forth in SEQ ID NO:6.

4. A recombinant nucleic acid comprising a nucleotide sequence encoding the polypeptide according to claim 1.

5. The recombinant nucleic acid according to claim 4 comprising a nucleotide sequence as set forth in SEQ ID NO:3 or SEQ ID NO:1.

6. A vector comprising the recombinant nucleic acid according to claim 4.

7. A host cell comprising the recombinant nucleic acid according to claim 4.

8. The host cell according to claim 7, which is an oleaginous host cell, preferably an oleaginous alga or an oleaginous yeast.

9. A method for the production of α-olefins comprising culturing a host cell according to claim 7 under conditions suitable for the production of α-olefins by said host cell.

10. The method according to claim 9, wherein said α-olefins are C₉-C₁₅ α-olefins, preferably C₉ or C₁₁ α-olefins.

11. The method according to claim 9, wherein said host cell is cultivated in medium comprising C₁₀-C₁₆ free fatty acids, preferably C₁₀ or C₁₂ free fatty acids.

12. The method according to claim 9, wherein the host cell has further been genetically engineered to produce or overproduce C₁₀-C₁₆ free fatty acids, preferably C₁₀ or C₁₂ free fatty acids, preferably wherein the host cell comprises a recombinant nucleic acid encoding an enzyme involved in the production of free fatty acids with a carbon chain length comprised between 10 and 16 such as a thioesterase having activity on C₁₀ to C₁₆ acyl-ACP.

13. The method according to claim 9, further comprising the step of recovering the α-olefins from the host cell or the culture medium.

14. A method for the production of α-olefins comprising contacting a polypeptide according to claim 1 with free fatty acids, preferably C₁₀-C₁₆ free fatty acids, more preferably C₁₀ or C₁₂ free fatty acids.

15. A method for the production of poly-α-olefins comprising the following steps:
i) producing α-olefins according to a method according to claim 9; and
ii) performing an oligomerization reaction using the α-olefins obtained in step i) as monomer to produce an oligomer; and optionally,
iii) hydrogenating the oligomer produced in step ii).

16. The method according to claim 15, wherein the poly-α-olefins are C₂₇ or C₃₃ poly-α-olefins, wherein step i) comprises the production of C₉ or C₁₁ α-olefins, and wherein the oligomerization reaction in step ii) is a trimerization reaction.

17. (canceled)

18. A method for the production of a lubricant, said method comprising:
i) producing poly-α-olefins according to the method of claim 15; and
ii) formulating a lubricant using said poly-α-olefins as base oil together with additives and optionally other base oils.

19. A host cell comprising the vector according to claim 6.
